(19) United States
(12) Patent Application Publication
(10) Pub. No.: US 2011/0190461 A1
(43) Pub. Date: Aug. 4, 2011

(54) NOVEL HETEROCYCLIC AROMATIC COMPOUND AND POLYMER

(75) Inventors: Yasuyuki Kita, Kashiwara-shi (JP); Toshifumi Dohi, Takatsuki-shi (JP); Koji Morimoto, Nara-shi (JP); Yoshiyuki Morita, Tatsuno-shi (JP); Tetsuya Hosomi, Tatsuno-shi (JP); Setsuko Ishioka, Tatsuno-shi (JP)

(73) Assignee: NAGASE CHEMTEX CORPORATION, Osaka-shi, Osaka (JP)

(21) Appl. No.: 13/054,425

(22) PCT Filed: Sep. 10, 2008
(86) PCT No.: PCT/JP2008/002506

§ 371 Date: Apr. 22, 2011

(30) Foreign Application Priority Data
Jul. 16, 2008 (JP) 2008-185327
Jul. 16, 2008 (JP) 2008-185328

(51) Int. Cl. C08F 34/04; C07D 495/04; C07D 491/056

(52) U.S. Cl. 526/256; 548/526; 548/518; 549/50

(57) ABSTRACT

A heterocycle-containing aromatic compound represented by the formula: A-B, and an electrically conductive polymer obtained by oxidative polymerization of the heterocycle-containing aromatic compound as a monomer are provided. In the above formula, A represents a substituted or unsubstituted thiophene ring group, or a substituted or unsubstituted pyrrole ring group; B represents a substituted or unsubstituted hydrocarbon aromatic ring group, a substituted or unsubstituted thiophene ring group, or a substituted or unsubstituted pyrrole ring group; the ring represented by A and the ring represented by B are directly linked; however, A and B represent structures that are different from each other. The compound can be produced by a coupling reaction using a hypervalent iodine reactant.

### TECHNICAL FIELD

The present invention relates to a heterocycle-containing aromatic compound, a heterocycle-containing electrically conductive polymer obtained by polymerizing the compound, a method for producing the same, and an electrically conductive resin composition containing the polymer.

### BACKGROUND ART

Conjugated polymers referred to as electrically conductive polymers have attracted attention as new materials having breakthrough performances and functions beyond conventional wisdom. These have been developed as, for example, various types of novel functional elements such as electroluminescent (EL) elements, secondary batteries and capacitors, and some of them have been already utilized as industrial products. Typical examples of known conjugated polymers include polyacetylene, polyparaphenylene, polyphenylenevinylene, polyphenylenesulfide, polypyrrole, polythiophene, poly(3-methylthiophene), polyaniline, polyperinaphthalene, polyacrylonitrile, and the like. These have been used in the field of, for example, solid electrolytic capacitors and the like (e.g., see Patent Document 1). However, these materials do not necessarily have superior physical properties in all aspects of electric conductivity, heat resistance, weather resistance, transparency, formation processability (in particular, solubility in solvents), and thus have been unsatisfactory for use in an electronic material field in which a high level of physical properties are demanded with good balance.

Among the aforementioned various types of electrically conductive polymers, particularly polypyrrole and polythiophene are electrically conductive polymers on which industrial use as solid electrolytic capacitors, organic solar cells, organic light emitting elements and an electrically conductive film in place of ITO has been investigated due to advantages in terms of the electric conductivity and/or formation processability (see Patent Documents 1, 2 and 3).

Electric conductivity, heat resistance, weather resistance and formation processability (in particular, solubility in solvents) of polypyrrole and polythiophene can be adjusted to some extent by introducing an appropriate substituent. However, similarly to other electrically conductive polymers, they do not necessarily have superior physical properties in terms of all aspects, and to impart a plurality of characteristics at the same time is very difficult, and thus they are still unsatisfactory for a higher level of requirements in recent years.

Known methods for producing polypyrrole or polythiophene include a method in which pyrrole or thiophene is subjected to electrochemical oxidative polymerization (electrolytic polymerization), or a method in which chemical oxidative polymerization (chemical polymerization) is carried out using an oxidizing agent. Membranous electrically conductive polymers obtained by electrolytic polymerization are involved in problems of inferior mass productivity and economical efficiency, as well as low strength per se, and difficulty in formation processing resulting from insolubility and infusibility. Additionally, pyrrole or thiophene to which a substituent was introduced may have lowered polymerizability depending on the type of the substituent, and thus to give an electrically conductive polymer may be difficult.
Patent Document 1: JP-A No. 2008-91358
Patent Document 2: JP-A No. 2007-165093
Patent Document 3: JP-A No. 2007-329454

### DISCLOSURE OF THE INVENTION

### Problems to be Solved by the Invention

The present invention was made for solving the foregoing conventional problems, and an object thereof is to provide an electrically conductive polymer having a plurality of superior characteristics such as electric conductivity, heat resistance, weather resistance, formation processability (in particular, solubility in solvents) and transparency, as well as a heterocycle-containing aromatic compound which can be a precursor of the same. Another object of the present invention is to provide a method for producing the heterocycle-containing aromatic compound, a polymerizable composition containing the heterocycle-containing aromatic compound, a simple method for producing the electrically conductive polymer, and an electrically conductive resin composition containing the electrically conductive polymer.

### Means for Solving the Problems

The present inventors thoroughly investigated in order to solve the foregoing problems, and consequently found that a non-fused bicyclic aromatic compound having a heterocyclic skeleton of at least either one of a thiophene ring or a pyrrole ring can be a precursor of an electrically conductive polymer or a cured product having superior electric conductivity, heat resistance, weather resistance, solubility in solvents, formation processability, and transparency. Thus, the present invention was accomplished.

Accordingly, the present invention relates to a heterocycle-containing aromatic compound represented by the following general formula (1):

A-B (1)

(wherein, A represents a substituted or unsubstituted thiophene ring group, or a substituted or unsubstituted pyrrole ring group; B represents a substituted or unsubstituted hydrocarbon aromatic ring group, a substituted or unsubstituted thiophene ring group, or a substituted or unsubstituted pyrrole ring group; the ring represented by A and the ring represented by B are directly linked; however, A and B represent structures that are different from each other.)

Another aspect of the present invention relates to a method for producing a heterocycle-containing aromatic compound represented by the above general formula (1), the method including allowing a compound represented by the formula of: A-H to be coupled with a compound represented by the formula of: B-H in the presence of a hypervalent iodine reactant.

Still other aspect of the present invention relates to a polymerizable composition containing the heterocycle-containing aromatic compound and a dopant.

Additional aspect of the present invention relates to an electrically conductive polymer obtained by oxidative polymerization of the heterocycle-containing aromatic compound as a monomer.

Still another aspect of the present invention relates to a method for producing an electrically conductive polymer including carrying out oxidative polymerization by a chemical polymerization method using an oxidizing agent, with the heterocycle-containing aromatic compound as a monomer.

Yet another aspect of the present invention relates to an electrically conductive resin composition containing the electrically conductive polymer.

### Effects of the Invention

The electrically conductive polymer of the present invention has superior physical properties in connection with electric conductivity, heat resistance, weather resistance, formation processability (in particular, solubility in solvents), transparency, and the like. The heterocycle-containing aromatic compound of the present invention is advantageous in preparing an electrically conductive polymer or a cured product having such superior physical properties.

### BEST MODE FOR CARRYING OUT THE INVENTION

The heterocycle-containing aromatic compound of the present invention, a method for producing the same, and a polymerizable composition containing the compound are explained first.

The heterocycle-containing aromatic compound of the present invention is a compound represented by the following formula (1). This compound may be herein referred to as “heterocycle-containing aromatic compound (1)”.

A-B (1)

Wherein, A represents a substituted or unsubstituted thiophene ring group, or a substituted or unsubstituted pyrrole ring group; B represents a substituted or unsubstituted hydrocarbon aromatic ring group, a substituted or unsubstituted thiophene ring group, or a substituted or unsubstituted pyrrole ring group; however, A and B represent structures that are different from each other.

The thiophene ring group as referred to herein means a 2-thienyl group, and may have a substituent on the carbon atom.

The pyrrole ring group as referred to herein means a 2-pyrrolyl group, and may have a substituent on the carbon atom or the nitrogen atom.

Examples of the substituted thiophene ring group and substituted pyrrole ring group represented by A or B in the formula (1) include, e.g., the structures as in the following.

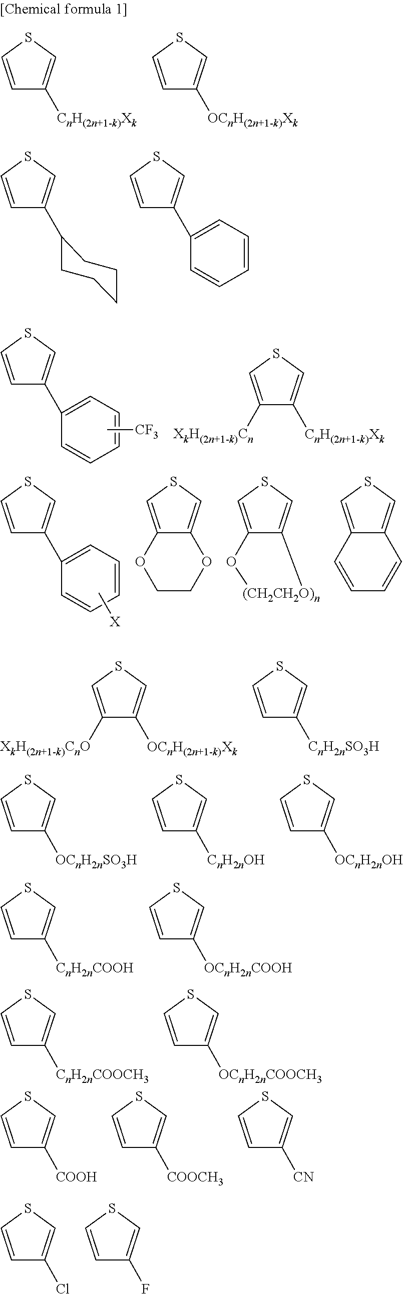

(In each formula, X represents a halogen atom; n is an integer of from 1 to 10; and k is an integer of from 0 to 20)

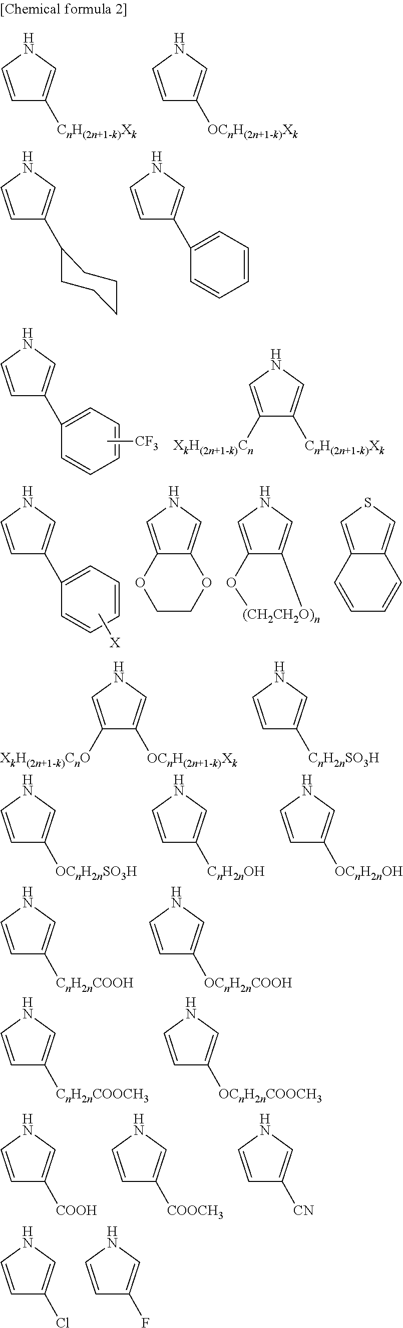

(In each formula, X represents a halogen atom; n is an integer of from 1 to 10; and k is an integer of from 0 to 20)

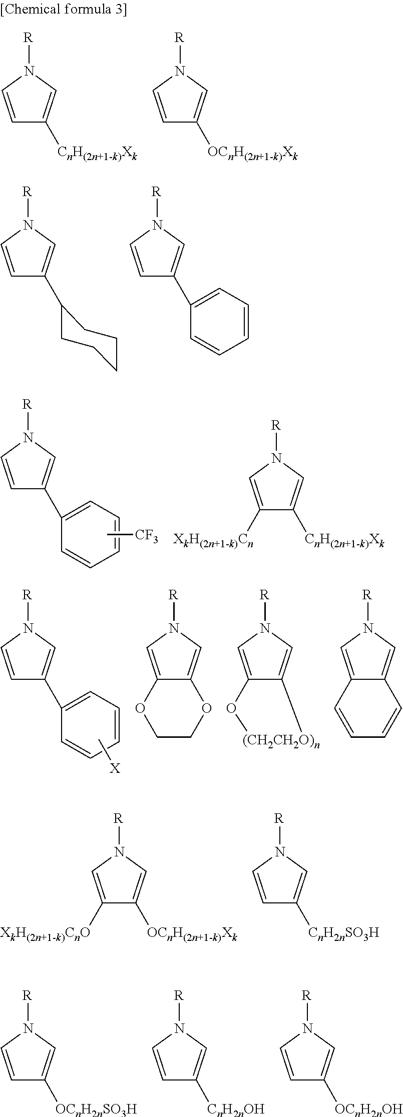

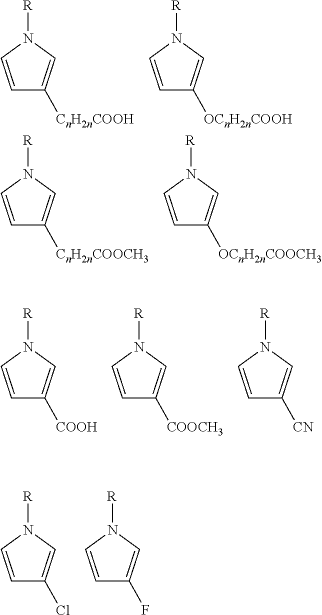

(In each formula, X represents a halogen atom; n is an integer of from 1 to 10; k is an integer of from 0 to 20; and R represents an aromatic group which may have a substituent, or an alkyl group having 1 to 10 carbon atoms.)

As the substituent on the thiophene ring group, organic groups described later are exemplified, and an alkyl group having 1 to 10 carbon atoms or an alkoxy group having 1 to 5 carbon atoms is preferred. In addition, as the substituent on the pyrrole ring group, organic groups described later are exemplified, and an alkyl group having 1 to 10 carbon atoms or an alkoxy group having 1 to 5 carbon atoms is preferred as the substituent on the carbon atom, whereas an alkyl group having 1 to 10 carbon atoms or a phenyl group which may have a substituent is preferred as the substituent on the nitrogen atom. To the alkyl group or alkoxy group introduced as a substituent of the thiophene ring group or the pyrrole ring group may be bound a halogen element, or a functional group such as a carboxylic acid group or a sulfonic acid group.

Although the hydrocarbon aromatic ring group is not particularly limited, for example, a phenyl group, a naphthyl group and the like are exemplified. The hydrocarbon aromatic ring group is preferably a phenyl group. These groups may have a substituent, and the substituent is exemplified by the organic groups described later. In particular, an alkyl group having 1 to 10 carbon atoms or an alkoxy group having 1 to 5 carbon atoms is preferred.

The ring represented by A and the ring represented by B are not linked via an atom not included in the ring structure, but directly linked via a binding between atoms included in both rings.

The heterocycle-containing aromatic compound (1) is preferably a compound in which the total number of the substituents bound to position 3 or position 4 of the thiophene ring group or the pyrrole ring group is at least 2, in light of solubility in solvents as well as heat resistance and weather resistance. In addition, when the total number of the substituents bound to position 3 or position 4 is 4 (i.e., when substituents are bound at all positions 3 and positions 4), the substituent at position 3 and the substituent at position 4 in at least one of A or B preferably bind to form a ring structure in order to avoid the steric hindrance.

The heterocycle-containing aromatic compound (1) is preferably a heterocycle-containing aromatic compound represented by any one of the following formulae (2) to (6), and in particular, a heterocycle-containing aromatic compound represented by the formula (2) or (3) including a thiophene ring group and a pyrrole ring group is particularly preferred.

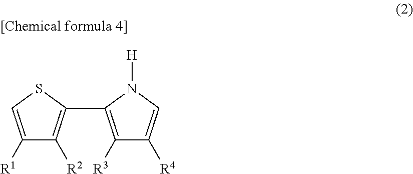

According to the formula (2), A in the formula (1) represents a thiophene ring group which may have substituent(s) at position 3 and/or position 4, and B represents a pyrrole ring group which may have substituent(s) at position 3 and/or position 4.

In the formula (2), R¹ and R² each independently represent a hydrogen atom or an organic group and at least one represents an organic group, and R³ and R⁴ each independently represent a hydrogen atom or an organic group (case i). Alternatively, R¹ and R² both represent a hydrogen atom; and R³ and R⁴ each independently represent an organic group (case ii). When both R⁴ and R² represent an organic group, these may bind with each other to form a ring structure. When both R³ and R⁴ represent an organic group, these may bind with each other to form a ring structure. As such a ring structure, a ring structure formed by an ethylenedioxy group is exemplified.

Preferably, in the formula (2), R¹ and R² each independently represent an organic group, which bind with each other to form a ring structure in the case i, and/or R³ and R⁴ each independently represent an organic group, which bind with each other to form a ring structure in the case ii.

More preferably, in the case i, R¹ and R² each independently represent an organic group, which bind with each other to form a ring structure.

Even more preferably, in light of achieving superior electric conductivity, R¹ and R² bind with each other to form an ethylenedioxy group in the case i. In this instance, the compound represented by the formula (2) is represented by the following formula (2′).

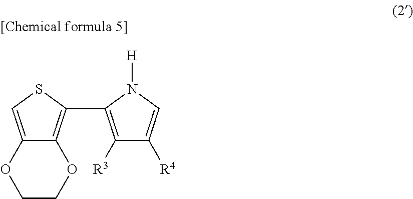

Particularly, the compound represented by the following formula, i.e., the compound represented by the formula (2′) in which R³ and R⁴ are a hydrogen atom is preferred.

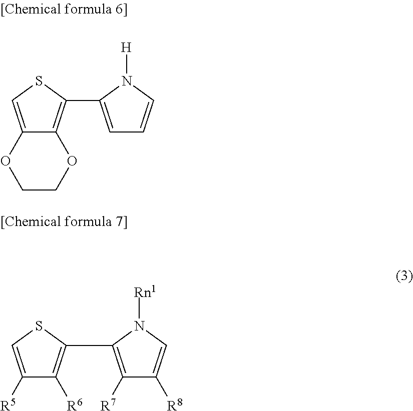

According to the formula (3), A in the formula (1) represents a thiophene ring group which may have substituent(s) at position 3 and/or position 4, and B represents an N-substituted pyrrole ring group which may have substituent(s) at position 3 and/or position 4.

In the formula (3), R⁵ and R⁶ each independently represent a hydrogen atom or an organic group and at least one represents an organic group, and R⁷ and R⁸ each independently represent a hydrogen atom or an organic group (case i). Alternatively, R⁵ and R⁶ both represent a hydrogen atom, and R⁷ and R⁸ each independently represent an organic group (case ii). When both R⁵ and R⁶ represent an organic group, these may bind with each other to form a ring structure. When both R⁷ and R⁸ represent an organic group, these may bind with each other to form a ring structure. As such a ring structure, a ring structure formed by an ethylenedioxy group is exemplified. Rn¹ represents an organic group.

Preferably, in the formula (3), R⁵ and R⁶ each independently represent an organic group, which bind with each other to form a ring structure in the case i, and/or R⁷ and R⁸ each independently represent an organic group, which bind with each other to form a ring structure in the case ii.

More preferably, in the formula (3), R⁵ and R⁶ each independently represent an organic group, which bind with each other to form a ring structure in the case i.

Still more preferably, in light of superior electric conductivity, R⁵ and R⁶ bind with each other to form an ethylenedioxy group in the case i. In this instance, the compound represented by the formula (3) is represented by the following formula (3′).

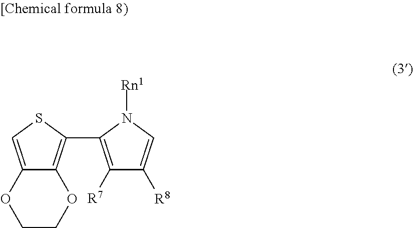

Particularly, a compound represented by the following formula (3″), i.e., the compound represented by the formula (3′) in which R⁷ and R⁸ represent a hydrogen atom, and Rn¹ represents a phenyl group which may have a substituent, or a compound represented by the following formula (3′″) in which R⁷ and R⁸ represent a hydrogen atom, and Rn¹ represents a naphthyl group is preferred.

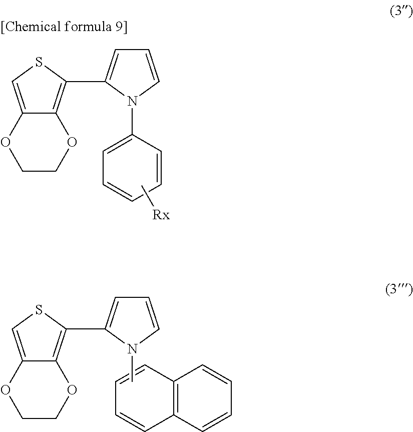

Rx in the formula (3″) represents a hydrogen atom, an organic group or a halogen atom. In light of the electric conductivity and solubility in solvents, a hydrogen atom, a fluorine atom, a methoxy group (—OCH₃), a trifluoromethyl group (—CF₃) and a methoxycarbonyl group (—COOCH₃) are particularly preferred.

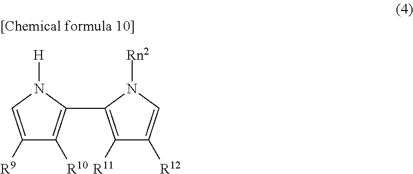

According to the formula (4), A in the formula (1) represents a pyrrole ring group which may have substituent(s) at position 3 and/or position 4, and B represents an N-substituted pyrrole ring group which may have substituent(s) at position 3 and/or position 4.

In the formula (4), R⁹ and R¹⁰ each independently represent a hydrogen atom or an organic group and at least one represents an organic group, and R¹¹ and R¹² each independently represent a hydrogen atom or an organic group (case i). Alternatively, R⁹ and R¹⁰ both represent a hydrogen atom, and R¹¹ and R¹² each independently represent an organic group (case ii). When both R⁹ and R¹⁰ represent an organic group, these may bind with each other to form a ring structure. When both R¹¹ and R¹² represent an organic group, these may bind with each other to form a ring structure. As such a ring structure, a ring structure formed by an ethylenedioxy group is exemplified. Rn² represents an organic group.

Preferably, in the formula (4), R⁹ and R¹⁰ each independently represent an organic group, which bind with each other to form a ring structure in the case i, and/or R¹¹ and R¹² each independently represent an organic group, which bind with each other to form a ring structure in the case ii.

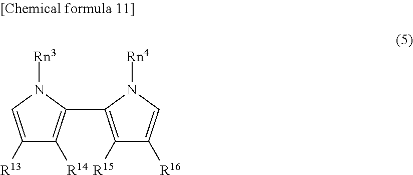

According to the formula (5), A and B in the formula (1) each independently represent a pyrrole ring group which may have substituent(s) at position 3 and/or position 4; however, since A and B represent structures that are different from each other, the combination of R¹³, R¹⁴ and Rn³ should not be identical to the combination of R¹⁵, R¹⁶ and Rn⁴.

In the formula (5), R¹³ and R¹⁴ each independently represent a hydrogen atom or an organic group and at least one represents an organic group, and R¹⁵ and R¹⁶ each independently represent a hydrogen atom or an organic group (case i). Alternatively, R¹³ and R¹⁴ both represent a hydrogen atom, and R¹⁵ and R¹⁶ each independently represent an organic group (case ii). When both R¹³ and R¹⁴ represent an organic group, these may bind with each other to form a ring structure. When both R¹⁵ and R¹⁶ represent an organic group, these may bind with each other to form a ring structure. As such a ring structure, a ring structure formed by an ethylenedioxy group is exemplified. Rn³ and Rn⁴ each independently represent an organic group.

Preferably, in the formula (5), R¹³ and R¹⁴ each independently represent an organic group, which bind with each other to form a ring structure in the case i, and/or R¹⁵ and R¹⁶ each independently represent an organic group, which bind with each other to form a ring structure in the case ii.

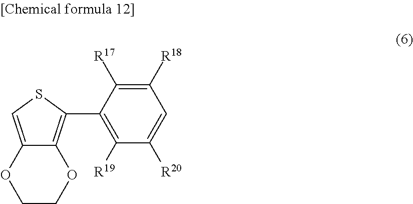

According to the formula (6), A in the formula (1) represents a 3,4-ethylenedioxythiophene ring group in which the substituent at position 3 and the substituent at position 4 on the thiophene ring bind to form an ethylenedioxy group, and B represents a phenyl group which may have substituent(s) at ortho-position and/or meta-position.

In the formula (6), R¹⁷ and R¹⁸ each independently represent a hydrogen atom or an organic group, and at least one represents an organic group. R¹⁹ and R²⁰ each independently represent an organic group. Since the ortho-position and the meta-position of a benzene ring are particularly easily oxidized, stability of the compound can be improved by allowing a substituent to bind to at least 3 of these positions.

As the organic group which may be represented by R¹ to R²⁰ and Rn¹ to Rn⁴, and Rx, for example, linear, branched or cyclic alkyl groups having 1 to 10 carbon atoms (e.g., a methyl group, an ethyl group, a propyl group, an isopropyl group, a butyl group, a s-butyl group, a t-butyl group, a hexyl group, a cyclohexyl group, etc.), linear, branched or cyclic alkenyl groups having 1 to 10 carbon atoms (e.g., an ethylene group, a propylene group, a butane-1,2-diyl group, a cyclohexenyl group, etc.), alkoxy groups having 1 to 5 carbon atoms (e.g., a methoxy group, an ethoxy group, an isopropoxy group, etc.), phenyl groups which may have a substituent (e.g., a phenyl group, a tolyl group, a dimethylphenyl group, a biphenyl group, a cyclohexylphenyl group, a 4-trifluoromethylphenyl group, a 4-fluorophenyl group, a 4-methoxyphenyl group, a 4-carbomethoxyphenyl group, etc.), a naphthyl group, aralkyl groups (e.g., a benzyl group, a phenethyl group, etc.), alkoxycarbonyl groups (e.g., a methoxycarbonyl group, etc.) are exemplified. Furthermore, to these organic groups may be bound a functional group such as a carboxyl group, an amino group, a nitro group, a cyano group, a sulfonic acid group or a hydroxyl group, or a halogen element such as fluorine, chlorine, bromine or iodine. In addition, R¹ to R²⁰ may be a carboxyl group, an amino group, a nitro group, a cyano group, a sulfonic acid group, a hydroxyl group or a halogen element. The organic group as described above is each independently selected.

The organic group which may be represented by R¹ to R²⁰ is preferably an alkyl group having 1 to 10 carbon atoms or an alkoxy group having 1 to 5 carbon atoms. The organic group which may be represented by Rn¹ to Rn⁴ is preferably an alkyl group having 1 to 10 carbon atoms, or a phenyl group, and particularly preferably a phenyl group.

When R¹ to R²⁰ that are adjacent to each other (R¹ and R², R³ and R⁴, R⁵ and R⁶, R⁷ and R⁸, R⁹ and R¹⁰, R¹¹ and R¹², R¹³ and R¹⁴, R¹⁵ and R¹⁶, R¹⁷ and R¹⁸, and R¹⁹ and R²⁰) are both an organic group, and these bind one another to form a ring structure, the ring structure is not particularly limited, but an alicyclic structure having 2 to 10 carbon atoms is preferred. The alicyclic structure may include an oxygen atom, a silicon atom, a sulfur atom, a nitrogen atom or the like, and especially, an ring structure having an alkylenedioxy group that contains an oxygen atom is particularly preferred. Moreover, the alicyclic structure may be aromatic, and this means that A or B in the heterocycle-containing aromatic compound (1) has a ring-fused structure (e.g., isothianaphthene, etc.).

The heterocycle-containing aromatic compound (1) can be produced by allowing two types of heterocyclic aromatic compounds, or a heterocyclic aromatic compound and a hydrocarbon aromatic compound to be coupled in the presence of a hypervalent iodine reactant. The present inventors found that such a coupling reaction proceeds efficiently in the presence of a hypervalent iodine reactant at a ratio of 1:1

The hypervalent iodine reactant as referred to herein means a reactant containing an iodine atom in a hypervalent state of trivalent or pentavalent. Since a hypervalent iodine reactant has a property being likely to return to more stable octet state (monovalent iodine), it has a similar reactivity to heavy metal oxidizing agents such as lead (IV), thallium (III) and mercury (II). Furthermore, a hypervalent iodine reactant has lower toxicity and is superior in safety as compared with such heavy metal oxidizing agents.

The hypervalent iodine reactant which may be used in the method for production of the present invention is not particularly limited. As the hypervalent trivalent iodine reactant, for example, phenyliodinbis(trifluoroacetate) or (bis(trifluoroacetoxy)iodobenzene (hereinafter, may be referred to as PIFA)), phenyliodin diacetate (iodosobenzene diacetate (hereinafter, may be referred to as PIDA)), hydroxy(tosyloxy)iodobenzene, iodosylbenzene, and the like are exemplified. Structure formulae of these reactants are shown below.

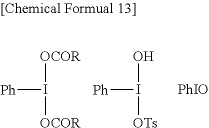

R═CH₃: Phenyliodin diacetate (PIDA)

### R═CF₃: Phenyliodinbistrifluoroacetate (PIFA)

Hydroxy(tosyloxy)iodobenzene (HTIB)

Iodosylbenzene

As the hypervalent pentavalent iodine reactant, for example, Dess-Martin periodinane (DMP), o-iodoxybenzoic acid (IBX), and the like are exemplified. The structure formulae of these reactants are shown below.

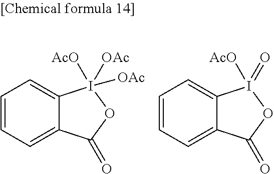

Dess-Martin periodinane (DMP), o-iodoxybenzoic acid (IBX)

Among these hypervalent iodine reactants, hypervalent trivalent iodine reactants are preferred, and PIFA is more preferred in terms of being stable and superior in handlability, and having sufficiently high oxidizing capacity.

Also, to select a hypervalent iodine reactant having an adamantane structure, or a hypervalent iodine reactant having a tetraphenylmethane structure among the hypervalent iodine reactants is preferred since it can be recovered and recycled. More specifically, a hypervalent iodine reactant having a trivalent adamantane structure such as 1,3,5,7-tetrakis-(4-(diacetoxyiodo)phenyl)adamantane, 1,3,5,7-tetrakis-((4-(hydroxy)tosyloxyiodo)phenyl)adamantane or 1,3,5,7-tetrakis-(4-bis(trifluoroacetoxyiodo)phenyl)adamantane, or a hypervalent iodine reactant having a trivalent tetraphenylmethane structure such as tetrakis-4-(diacetoxyiodo)phenylmethane or tetrakis-4-bis(trifluoroacetoxyiodo)phenylmethane is more preferred due to being stable and superior in handlability, and having sufficiently high oxidizing capacity, as well as being highly lipid soluble, and capable of being recovered and recycled. When a hypervalent pentavalent iodine reactant is used, Dess-Martin periodinane (DMP) is preferred.

As such a hypervalent iodine reactant, either a product obtained by synthesis, or a commercially available product may be used. For example, PIFA may be obtained by adding trifluoroacetic acid to PIDA to permit a reaction, and allowing thus resulting PIFA to be deposited as a reaction product (see, J. Chem. Soc. Perkin Trans. 1, 1985, 757). PIDA may be obtained by oxidizing iodobenzene in acetic acid using sodium peroxoborate (tetrahydrate) (NaBO₃.4H₂O) (see, Tetrahedron, 1989, 45, 3299 and Chem. Rev., 1996, 96, 1123). Furthermore, PIDA may be obtained from iodobenzene using m-chloroperbenzoic acid (mCPBA) as an oxidizing agent (see, Angew. Chem. Int. Ed., 2004, 43, 3595). 1,3,5,7-Tetrakis-(4-(diacetoxyiodo)phenyl)adamantane, 1,3,5,7-tetrakis-((4-(hydroxy)tosyloxyiodo)phenyl)adamantane, 1,3,5,7-tetrakis-(4-bis(trifluoroacetoxyiodo)phenyl)adamantane, tetrakis-4-(diacetoxyiodo)phenylmethane and tetrakis-4-bis(trifluoroacetoxyiodo)phenylmethane can be synthesized according to, for example, a method disclosed in JP-A No. 2005-220122.

In the method for production of the present invention, the amount of the hypervalent iodine reactant is not particularly limited, and the hypervalent iodine reactant may be used in an amount of preferably 0.1 to 4 mol, more preferably 0.2 to 3 mol, and still more preferably 0.3 to 2 mol relative to 1 mol of one type of the source material.

According to the method for production of the present invention, a compound A-H selected from the group consisting of a substituted or unsubstituted thiophene compound and a substituted or unsubstituted pyrrole compound, and a compound B-H selected from the group consisting of a substituted or unsubstituted hydrocarbon aromatic compound, a substituted or unsubstituted thiophene compound and a substituted or unsubstituted pyrrole compound are used as source materials. Herein, A and B are similar to those described above. These compounds may be selected ad libitum in order to obtain a desired product, and specifically, the compounds as in the following may be used.

As the thiophene compound which may be used in the method for production of the present invention, for example, thiophene, position 3-substituted thiophene, and positions 3,4-substituted thiophene are exemplified. Specific examples include thiophene, 3-methylthiophene, 3-hexylthiophene, 3-phenylthiophene, 3,4-ethylenedioxythiophene, 3,4-propylenedioxythiophene, 3-methoxythiophene, 3-butoxythiophene, and the like. Among these, when a substituted thiophene is used, the type and the substitution position of the substituent are not particularly limited, but it is preferred that a substituted thiophene having an alkyl group or an alkoxy group at position 3 and position 4 be used.

As the pyrrole compound which may be used in the method for production of the present invention, for example, pyrrole, position 3-substituted pyrrole, positions 3,4-substituted pyrrole, and N-substituted pyrrole are exemplified. Specific examples include pyrrole, 3-methylpyrrole, 3-hexylpyrrole, 3-phenylpyrrole, N-phenylpyrrole, N-ethylsulfonate pyrrole, 3,4-cyclohexylpyrrole, and the like. Among these, when a substituted pyrrole is used, the type and the substitution position of the substituent are not particularly limited, but it is preferred that a substituted pyrrole having an alkyl group or aryl group at N-position, particularly a substituted pyrrole having a phenyl group which may have a substituent or a naphthyl group at N-position be used. Examples of such a substituted pyrrole include N-(4-fluorophenyl)pyrrole, N-(4-chlorophenyl)pyrrole, N-(4-cyanophenyl)pyrrole, N-(4-nitrophenyl)pyrrole, N-(4-aminophenyl)pyrrole, N-(4-methoxyphenyl)pyrrole, N-(4-(1-oxoethyl)phenyl)pyrrole, N-(4-trifluoromethylphenyl)pyrrole, N-(4-carbomethoxyphenyl)pyrrole, N-(4-carboxyphenyl)pyrrole, N-(1-naphthyl)pyrrole, N-(2-naphthyl)pyrrole, and the like.

As the hydrocarbon aromatic compound which may be used in the method for production of the present invention, benzene-based aromatic compounds such as benzene, toluene, p-dimethoxy benzene and cresol, polycyclic aromatic compounds such as biphenyl and triphenylmethane, aromatic condensed-ring compounds such as naphthalene and anthracene, and the like are exemplified. In particular, benzene-based aromatic compounds are preferred, and benzene or 1,4-substituted benzene is particularly preferred.

The coupling reaction in the method for production of the present invention is usually carried out in the presence of a solvent. The solvent which can be used in the method for production of the present invention may be any solvent that dissolves or disperses the source material, and the hypervalent iodine reactant. Examples of such a solvent include water, organic solvents (alcohols such as methanol, ethanol, 2-propanol, 1-propanol and n-butanol; ethylene glycols such as ethylene glycol, diethylene glycol, triethylene glycol and tetraethylene glycol; glycol ethers such as ethylene glycol monomethyl ether, diethylene glycol monomethyl ether, ethylene glycol dimethyl ether, ethylene glycol diethyl ether and diethylene glycol dimethyl ether; glycol ether acetates such as ethylene glycol monoethyl ether acetate, diethylene glycol monoethyl ether acetate and diethylene glycol monobutyl ether acetate; propylene glycols such as propylene glycol, dipropylene glycol and tripropylene glycol; propylene glycol ethers such as propylene glycol monomethyl ether, propylene glycol monoethyl ether, dipropylene glycol monomethyl ether, dipropylene glycol monoethyl ether, propylene glycol dimethyl ether, dipropylene glycol dimethyl ether, propylene glycol diethyl ether and dipropylene glycol diethyl ether; propylene glycol ether acetates such as propylene glycol monomethyl ether acetate, propylene glycol monoethyl ether acetate, dipropylene glycol monomethyl ether acetate and dipropylene glycol monoethyl ether acetate; N-methylformamide, N,N-dimethyl formamide, N-methylpyrrolidone, dimethylacetamide, dimethyl sulfoxide, acetone, acetonitrile, and toluene, xylene (o-, m-, or p-xylene), benzene, ethyl acetate, butyl acetate, methyl ethyl ketone, methyl isobutyl ketone, diethyl ether, diisopropyl ether, methyl-t-butyl ether, hexane, heptane, chloromethane (methyl chloride), dichloromethane (methylene chloride), trichloromethane (chloroform) and tetrachloromethane (carbon tetrachloride), etc.), as well as mixed solvents of water and any of these organic solvents (aqueous organic solvents), and mixed solvents of two or more kinds of the organic solvents.

According to the method for production of the present invention, an additive may be appropriately added into the coupling reaction system. By using the hypervalent iodine reactant and the additive in combination, the yield of the heterocycle-containing aromatic compound can be improved, and the amount of the hypervalent iodine reactant can be reduced. Examples of the additive include, e.g., bromotrimethylsilane, chlorotrimethylsilane, trimethylsilyl triflate, boron trifluoride, trifluoroacetic acid, hydrochloric acid, sulfuric acid and the like, and bromotrimethylsilane is particularly preferred. These may be used alone, or a plurality of these may be used. With respect to the amount of the additive, the additive may be used in an amount of preferably 0.1 to 4 mol, more preferably 0.2 to 3 mol, and still more preferably 0.5 to 2 mol relative to 1 mol of one type of the source material.

According to the method for production of the present invention, a fluorine-based alcohol may be added into the coupling reaction system. By using the hypervalent iodine reactant and the fluorine-based alcohol in combination, the yield of the heterocycle-containing aromatic compound can be improved, and the amount of the hypervalent iodine reactant can be reduced. As the fluorine-based alcohol which may be added, for example, 1,1,1,3,3,3-hexafluoro-2-propanol, trifluoroethanol, hexafluoroethanol and the like are exemplified, and 1,1,1,3,3,3-hexafluoro-2-propanol is particularly preferred. Although the amount of the fluorine-based alcohol is not particularly limited, it is preferably 1 to 80 parts by weight, and particularly preferably 10 to 40 parts by weight relative to 100 parts by weight of the solvent employed.

The coupling reaction is carried out, in general, in the temperature range of −50° C. to 100° C. for 10 min to 48 hrs after mixing each source material, the hypervalent iodine reactant and the solvent as well as other reagents, etc., thereby capable of producing the heterocycle-containing aromatic compound (1).

Preferably, the coupling reaction is carried out in the temperature range of 0 to 50° C. for 30 min to 8 hrs. More preferably, the coupling reaction is carried out in the temperature range of 10 to 40° C. for 1 to 4 hrs. The reagents may be added in any order.

The polymerizable composition of the present invention contains the heterocycle-containing aromatic compound (1), and a dopant. The polymerizable composition refers to a composition which can provide thin membranes, films, etc., of an electrically conductive polymer by polymerization of the heterocycle-containing aromatic compound (1) via an action of an oxidizing agent or oxygen in the air. By applying this polymerizable composition onto various types of base materials (plastic substrates, glass substrates, and metals such as oxidized film-having aluminum and sintered tantalum, used for producing solid electrolytic capacitors), an electrically conductive thin membrane or film can be conveniently formed. As the heterocycle-containing aromatic compound (1), only one type may be used, or two types or more may be used as a mixture. It is preferred that the polymerizable composition of the present invention contains the heterocycle-containing aromatic compound (1) in an amount of 10 to 90% by weight in the total amount of the composition.

The polymerizable composition of the present invention may further contain in addition to the dopant (i), (ii) an oxidizing agent, (iii) a binder resin, (iv) an additive, (v) a solvent, and the like as needed.

The aforementioned dopant (i) is an electron donating or acceptable chemical substance which acts on the electrically conductive polymer generated by polymerization of the heterocycle-containing aromatic compound (1) due to the action of the oxidizing agent or oxygen in the air, and thus can dramatically improve the electric conductivity thereof. Although the dopant is not particularly limited, as acceptors (p-dopants) that inject holes into electrically conductive polymers to permit oxidization: halogen such as Cl₂, Br₂, I₂ and ICl; Lewis acids such as PF₅, BF₃ and trimethylsilyl trifluoromethane sulfonate; protonic acids such as HF, HCl, HNO₃ and H₂SO₄; organic acids such as p-toluenesulfonic acid and polystyrene sulfonic acid, and the like are exemplified. As donors (n-dopants) that inject electrons to permit reduction: alkali metals such as lithium, sodium, rubidium and cesium; alkaline earth metals such as beryllium, magnesium, calcium, scandium and barium; silver, europium, ytterbium, and the like are exemplified. These dopants may be used at a rate of preferably 0.01 to 20 mol, and more preferably 0.5 to 10 mol, relative to 1 mol of the heterocycle-containing aromatic compound (1).

The aforementioned oxidizing agent (ii) is contained for the purpose of accelerating polymerization of the heterocycle-containing compound (1), and examples thereof include iron (III)-based compounds such as p-toluenesulfonic acid iron (III), dodecyl benzenesulfonic acid iron (III), benzenesulfonic acid iron (III), methanesulfonic acid iron (III), ethanesulfonic acid iron (III), α-sulfo-naphthalene iron (III), β-sulfo-naphthalene iron (III), naphthalenedisulfonic acid iron (III) and alkylnaphthalenesulfonic acid iron (III) (wherein, the alkyl group may be butyl, triisopropyl, di-t-butyl or the like), etc., perchloric acid iron (III) and iron chloride (III), as well as anhydrous aluminum chloride/cuprous chloride, alkali metal persulfuric acid salts, persulfuric acid ammonium salts, peroxides, manganeses such as potassium permanganate, quinones such as 2,3-dichloro-5,6-dicyano-1,4-benzoquinone (DDQ), tetrachloro-1,4-benzoquinone and tetracyano-1,4-benzoquinone, harogens such as iodine and bromine, peracid, sulfonic acid such as sulfuric acid, fuming sulfuric acid, sulfur trioxide, chlorosulfuric acid, fluorosulfuric acid, amidesulfuric acid; ozone, etc., and combinations of these pluralities of oxidizing agents. Of these, p-toluenesulfonic acid iron (III), dodecyl benzenesulfonic acid iron (III) and iron chloride (III) are particularly preferred since they also serve as the aforementioned dopant (i) due to containing sulfonic acid or halogen. The polymerizable composition of the present invention enables polymerization to proceed with oxygen in the air even if the aforementioned oxidizing agent is not added; however, the oxidizing agent may be also added if necessary. When such an oxidizing agent is added, the oxidizing agent is contained in an amount of preferably 0.01 to 10 mol, and more preferably 0.1 to 4 mol relative to 1 mol of the heterocycle-containing aromatic compound (1).

Although the aforementioned binder resin (iii) is not particularly limited, polyesters, poly(meth)acrylates, polyurethanes, polyvinyl acetates, polyvinylidene chloride, polyamides, polyimides, and copolymers constituted with at least two monomers selected from the group consisting of styrene, vinylidene chloride, vinyl chloride and alkyl (meth)acrylate, and the like are exemplified. The polymerizable composition of the present invention contains the binder resin in an amount of preferably 10 to 5,000 parts by weight, and more preferably 20 to 3,000 parts by weight relative to 100 parts by weight of the heterocycle-containing aromatic compound (1).

As the aforementioned additive (iv), a silane coupling agent for improving adhesiveness with the substrate or for improving durability of the coated film, as well as a leveling agent or a surfactant for improving coating properties are exemplified. Examples of the silane coupling agent include 3-glycidoxypropyltrimethoxysilane, 3-glycidoxypropyltriethoxysilane, 2-(3,4-epoxycyclohexyl)methyltrimethoxysilane, 2-(3,4-epoxycyclohexyl)methyltriethoxysilane, 2-(3,4-epoxycyclohexyl)ethyltrimethoxysilane, 2-(3,4-epoxycyclohexyl)ethyltriethoxysilane, (meth) acryloxytrialkoxysilane such as 3-methacryloxypropyltriethoxysilane, 3-methacryloxypropyltrimethoxysilane, 3-acryloxypropyltrimethoxysilane and 3-acryloxypropyltriethoxysilane; vinyltrimethoxysilane, vinyltriethoxysilane, p-styryltrimethoxysilane, and the like. These may be used alone, or a plurality of these may be used. When the silane coupling agent is compounded in the polymerizable composition of the present invention, the silane coupling agent is contained in an amount of preferably 0.1 to 1,000 parts by weight, and more preferably 1 to 500 parts by weight relative to 100 parts by weight of the heterocycle-containing aromatic compound (1). Examples of the surfactant include nonionic surfactants (e.g., polyoxyethylene alkylphenyl ethers, polyoxyethylene alkyl ethers, sorbitan fatty acid esters, fatty acid alkylolamides, and the like), fluorochemical surfactants (e.g., fluoroalkylcarboxylic acids, perfluoroalkylbenzenesulfonic acids, perfluoroalkyl quaternary ammoniums, perfluoroalkylpolyoxyethylene ethanols, and the like). The polymerizable composition of the present invention contains preferably 0.01 to 80 parts by weight, and more preferably 0.05 to 30 parts by weight of the additive relative to 100 parts by weight of the polymerizable composition.

Although the aforementioned solvent (v) is not particularly limited, water, organic solvents (alcohols such as methanol, ethanol, 2-propanol, 1-propanol and n-butanol; ethylene glycols such as ethylene glycol, diethylene glycol, triethylene glycol and tetraethylene glycol; glycol ethers such as ethylene glycol monomethyl ether, diethylene glycol monomethyl ether, ethylene glycol dimethyl ether, ethylene glycol diethyl ether and diethylene glycol dimethyl ether; glycol ether acetates such as ethylene glycol monoethyl ether acetate, diethylene glycol monoethyl ether acetate and diethylene glycol monobutyl ether acetate; propylene glycols such as propylene glycol, dipropylene glycol and tripropylene glycol; propylene glycol ethers such as propylene glycol monomethyl ether, propylene glycol monoethyl ether, dipropylene glycol monomethyl ether, dipropylene glycol monoethyl ether, propylene glycol dimethyl ether, dipropylene glycol dimethyl ether, propylene glycol diethyl ether and dipropylene glycol diethyl ether; propylene glycol ether acetates such as propylene glycol monomethyl ether acetate, propylene glycol monoethyl ether acetate, dipropylene glycol monomethyl ether acetate and dipropylene glycol monoethyl ether acetate; N-methylformamide, N,N-dimethyl formamide, N-methylpyrrolidone, dimethylacetamide, dimethyl sulfoxide, acetone, acetonitrile, and toluene, xylene (o-, m-, or p-xylene), benzene, ethyl acetate, butyl acetate, methyl ethyl ketone, methyl isobutyl ketone, diethyl ether, diisopropyl ether, methyl-t-butyl ether, hexane, heptane, chloromethane (methyl chloride), dichloromethane (methylene chloride), trichloromethane (chloroform) and tetrachloromethane (carbon tetrachloride), etc.), as well as mixed solvents of water and any of these organic solvents (aqueous organic solvents), and mixed solvents of two or more kinds of the organic solvents are exemplified. The polymerizable composition of the present invention contains preferably 100 to 10,000 parts by weight, and more preferably 1,000 to 6,000 parts by weight of the solvent relative to 100 parts by weight of the heterocycle-containing aromatic compound (1).

The polymerizable composition of the present invention is cured by, for example, developing to give a thin membranous state having a thickness of about 0.1 to 30 μm, followed by, for example, heating at about 50 to 200° C., or irradiating rays of light, etc., and then dried if necessary, thereby capable of obtaining an electric conductivity thin membrane or film.

Next, the electrically conductive polymer of the present invention, a method for producing the same, and a resin composition containing the polymer are explained.

The heterocycle-containing aromatic polymer of the present invention is an electrically conductive polymer obtained by oxidative polymerization using as a monomer a non-fused bicyclic aromatic compound represented by the above general formula (1) having a heterocyclic skeleton of at least either one of a thiophene ring or a pyrrole ring.

By using different kinds of a heterocyclic aromatic compound and a hydrocarbon aromatic compound in combination, different properties derived from two constitutive elements can be imparted to the electrically conductive polymer; therefore, a high level of physical properties can be attained, which is not attainable with conventional polymers produced using a single compound as a monomer. Moreover, by appropriately selecting the structures of the different types of the heterocyclic aromatic compound and the hydrocarbon aromatic compound, not only adjustment of solubility in solvents and transparency, but also adjustment of the level of the electric conductivity is enabled to meet the purpose of use. Accordingly, industrially applicable range of the electrically conductive polymer can be expanded. In addition, by combining the thiophene structure and the pyrrole structure, a polymer that is superior in the electric conductivity, as well as heat resistance, weather resistance, solubility in solvents, and formation processability is provided.

Moreover, since the electrically conductive polymer of the present invention is obtained by polymerization of the non-fused bicyclic aromatic compound, the unit derived from A, and the unit derived from B are included in the polymer at a ratio of about 1:1. Therefore, unlike the case in which two types of monocyclic compounds are subjected to random polymerization, strict control of the ratio of two units is enabled. In addition, since the compound represented by A-B is used as a monomer, the repeating unit derived from A, and the repeating unit derived from B can almost nuiformly distribute in the polymer without deviation, whereby the polymer can exhibit homogenous physical properties.

The heterocycle-containing aromatic polymer of the present invention may be herein referred to as “heterocycle-containing aromatic polymer (1)”.

Examples of the substituted thiophene ring group and the substituted pyrrole ring group represented by A or B in the formula (1) include e.g., the structures as described in the foregoing.

In the monomer of the heterocycle-containing aromatic polymer (1), at least one of carbon atoms on the ring represented by A is nonsubstituted, and at least one of carbon atoms on the ring represented by B is nonsubstituted. When such a monomer is subjected to oxidative polymerization, a coupling reaction proceeds between unsubstituted carbon atoms, whereby a linear polymer having the repeating unit represented by -A-B- is obtained as the heterocycle-containing aromatic polymer (1). It is preferred that the thiophene ring or the pyrrole ring represented by A or B bind with each other between carbon atoms on a position 2, whereas the carbon atoms on the other position 2 are nonsubstituted.

As the monomer of the heterocycle-containing aromatic polymer (1), the heterocycle-containing aromatic compounds represented by any one of the formulae (2) to (6) described above are preferred, and of these, the heterocycle-containing aromatic compound represented by the formula (2) or (3) which includes a thiophene ring group and a pyrrole ring group is particularly preferred. According to these monomers, the oxidative polymerization proceeds at unsubstituted carbon atom of the position 2 on the thiophene ring, as well as at the unsubstituted carbon atom of the position 2 on the pyrrole ring.

The method for producing the heterocycle-containing aromatic polymer of the present invention (1) is characterized by subjecting the monomer to oxidative polymerization carried out by a chemical polymerization method using a variety of types of oxidizing agent. Since the chemical polymerization method enables large-scale production in a convenient manner, the method is more suited for industrial production process as compared with conventional electrolytic polymerization method.

The oxidizing agent for use in the chemical polymerization method is not particularly limited. Preferred oxidizing agents are exemplified by oxidizing agents including a sulfonic acid compound as an anion, and a transition metal having a high valence as a cation. As the transition metal ion having a high valence that constitutes this oxidizing agent, Ag⁺, Cu²⁺, Fe³⁺, Al³⁺, Ce⁴⁺, W⁶⁺, Mo⁶⁺, Cr⁶⁺, Mn⁷⁺, and Sn⁴⁺ are exemplified. In particular, Fe³⁺ and Cu²⁺ are preferred. Specific examples include FeCl₃, Fe(ClO₄)₃, K₂CrO₇, alkali or ammonium persulfate, alkali perborate, potassium permanganate, copper tetrafluoroborate. In addition, as oxidizing agents not including a metal ion, H₂O₂ and ammonium persulfate are exemplified. Still further, hypervalent compounds typified by hypervalent iodine reactants may be included.

In a particularly preferred embodiment, the oxidizing agent is a hypervalent iodine reactant. The hypervalent iodine reactant is similar to those described above.

The amount of the oxidizing agent used in the method for production of the present invention is not particularly limited, but preferably falls within the range of 1 to 5 mol, and more preferably falls within the range of 2 to 4 mol per mol of the monomer. In particular, when a hypervalent iodine reactant is used as the oxidizing agent, it is used at a rate of preferably 1 to 4 mol, more preferably 1.5 to 4 mol, and still more preferably 2 to 2.5 mol per mol of the monomer. When the amount of the hypervalent iodine reactant is small, it may be difficult to allow the oxidization polymerization reaction to proceed. On the other hand, when the amount of the hypervalent iodine reactant is too large, excess oxidization occurs, whereby a product that is in any way not soluble in the solvent may be obtained, and thus the yield of the desired polymer may be lowered.

In the method for production of the present invention, the hypervalent iodine reactant and an oxidizing agent not including a metal may be used in combination. By using the hypervalent iodine reactant with an oxidizing agent not including a metal in combination, the amount of the hypervalent iodine reactant can be reduced. As the oxidizing agent not including a metal, for example, peroxodisulfuric acid, ammonium peroxodisulfate, hydrogen peroxide, metachloroperbenzoic acid, and the like are exemplified.

When the hypervalent iodine reactant and the oxidizing agent not including a metal are used in combination, the hypervalent iodine reactant serves as an oxidization catalyst, and may be used at a rate of preferably 0.001 to 0.3 mol, and more preferably 0.01 to 0.1 mol per mol of the monomer. On the other hand, the oxidizing agent not including a metal is used at a rate of preferably 1 to 4 molar equivalents, and more preferably 1.5 to 2.5 molar equivalents per mol of the monomer.

When the oxidizing agent not including a metal and the hypervalent iodine reactant are used in combination, too small amount of the hypervalent iodine reactant may lead to failure in allowing the polymerization reaction to satisfactorily proceed. On the other hand, too large amount of the hypervalent iodine reactant may lead to waste of the hypervalent iodine reactant as the degree of polymerization is not elevated to exceed a certain level.

It is to be noted that when the hypervalent iodine reactant and the oxidizing agent not including a metal are used in combination, a precursor of a hypervalent iodine reactant may be used upon initiation of the polymerization reaction. For example, the hypervalent iodine reactant may be generated in the reaction system by adding a catalytic amount of 1,3,5,7-tetrakis-(4-iodophenyl)adamantane that is a precursor of 1,3,5,7-tetrakis-(4-(diacetoxyiodo)phenyl)adamantane, and a stoichiometric amount of metachloroperbenzoic acid.

The heterocycle-containing aromatic polymer (1) obtained by the method for production of the present invention may be doped with a dopant. By doping with a dopant, electric conductivity can be imparted to the obtained heterocycle-containing aromatic polymer (1). The dopant may be charged as a source material before carrying out the polymerization reaction, may be added during the polymerization reaction, or may be added to the heterocycle-containing aromatic polymer obtained after the polymerization reaction.

Although the dopant is not particularly limited, halogens such as Cl₂, Br₂, I₂ and ICl; Lewis acids such as PF₅, BF₃ and trimethylsilyl trifluoromethanesulfonate; protonic acids such as HF, HCl, HNO₃ and H₂SO₄; organic acids such as p-toluenesulfonic acid and polystyrenesulfonic acid, and the like are exemplified.

The dopant used for the purpose of imparting electric conductivity is contained at a rate of preferably 0.05 to 6 mol, and more preferably 0.2 to 4 mol per mol of the monomer.

When the amount of the dopant is less than 0.05 mol, sufficient electric conductivity may not be imparted to the heterocycle-containing aromatic polymer (1). On the other hand, when the amount of the dopant is greater than 6 mol, the entire dopant added to the heterocycle-containing aromatic polymer (1) is not doped, and thus the effect corresponding to the amount of the added dopant is not expected. Also, excess dopant is wasteful.

It should be noted that the Lewis acid not only serves as a dopant, but also has an effect of promoting the oxidization polymerization reaction. When the Lewis acid is used for the purpose of promoting the oxidization polymerization reaction, trimethylsilyl trifluoromethanesulfonate is particularly preferably used.

The oxidization polymerization reaction in the method for production of the present invention is usually carried out in the presence of a solvent. The solvent used in the method for production of the present invention is acceptable as long as it can dissolve or disperse the monomer, the oxidizing agent, and the dopant. Examples of such a solvent include water, organic solvents (alcohols such as methanol, ethanol, 2-propanol, 1-propanol and n-butanol; ethylene glycols such as ethylene glycol, diethylene glycol, triethylene glycol and tetraethylene glycol; glycol ethers such as ethylene glycol monomethyl ether, diethylene glycol monomethyl ether, ethylene glycol dimethyl ether, ethylene glycol diethyl ether and diethylene glycol dimethyl ether; glycol ether acetates such as ethylene glycol monoethyl ether acetate, diethylene glycol monoethyl ether acetate and diethylene glycol monobutyl ether acetate; propylene glycols such as propylene glycol, dipropylene glycol and tripropylene glycol; propylene glycol ethers such as propylene glycol monomethyl ether, propylene glycol monoethyl ether, dipropylene glycol monomethyl ether, dipropylene glycol monoethyl ether, propylene glycol dimethyl ether, dipropylene glycol dimethyl ether, propylene glycol diethyl ether and dipropylene glycol diethyl ether; propylene glycol ether acetates such as propylene glycol monomethyl ether acetate, propylene glycol monoethyl ether acetate, dipropylene glycol monomethyl ether acetate and dipropylene glycol monoethyl ether acetate; N-methylformamide, N,N-dimethyl formamide, N-methylpyrrolidone, dimethylacetamide, dimethyl sulfoxide, acetone, acetonitrile, and toluene, xylene (o-, m-, or p-xylene), benzene, ethyl acetate, butyl acetate, methyl ethyl ketone, methyl isobutyl ketone, diethyl ether, diisopropyl ether, methyl-t-butyl ether, hexane, heptane, chloromethane (methyl chloride), dichloromethane (methylene chloride), trichloromethane (chloroform) and tetrachloromethane (carbon tetrachloride), etc.), as well as mixed solvents of water and any of these organic solvents (aqueous organic solvents), and mixed solvents of two or more kinds of the organic solvents.

The temperature of the oxidization polymerization reaction of the present invention is preferably −100° C. to 100° C. In both cases in which an organic solvent is used as the solvent and water is used as the solvent, the temperature is more preferably 0° C. to 40° C. When the reaction temperature is lower than −100° C., the reaction velocity is lowered, and freezing may occur depending on the solvent employed, whereby the yield of the heterocycle-containing aromatic polymer (1) may be reduced. On the other hand, when the reaction temperature is higher than 100° C., a side reaction or excessive oxidization may occur, and thus the yield of the heterocycle-containing aromatic polymer (1) may be reduced.

In the method for production of the present invention, the reaction time of the oxidization polymerization reaction is not particularly limited. When a Lewis acid is used for promoting the oxidization polymerization reaction, the reaction time is preferably about 12 hrs, whereas when a Lewis acid is not used, the reaction time is preferably about 20 hrs.

Thus obtained heterocycle-containing aromatic polymer (1) may be purified. Although the process for purification (purification step) is not particularly limited, for example, a process in which the solvent is filtered with a glass filter after completing the reaction, and the obtained polymer is washed with methanol, ethanol, 2-propanol, n-hexane, diethyl ether, acetonitrile, ethyl acetate, toluene or the like. As the other purification process, purification with Soxhlet extraction or the like may be exemplified.

After washing, thus obtained heterocycle-containing aromatic polymer (1) is dried if necessary by common means (drying step). The drying process may be determined ad libitum depending on the degree of polymerization, the substituent, and the dopant contained. For example, drying at room temperature (about 25° C.) under reduced pressure (about 0.5 mmHg), drying under ordinary pressure with heated air blowing (about 60° C.), and the like are exemplified. The drying temperature is preferably not higher than 100° C., and the risk of degradation of the heterocycle-containing aromatic polymer (1) may increase when the temperature is higher than 200° C.

When the hypervalent iodine reactant having an adamantane structure or a tetraphenylmethane structure is used, the reactant is recovered by the process as in the following. For example, the solution after completing the reaction is concentrated under reduced pressure, and methanol is added to the residue (the polymer, the hypervalent iodine reactant having an adamantane structure or a tetraphenylmethane structure, the oxidizing agent not including a metal, and the unreacted monomer), followed by mixing, and then filtering the mixture using a glass filter, thereby capable of removing the oxidizing agent not including a metal and unreacted monomer in a methanol solution. To the polymer and the hypervalent iodine reactant having an adamantane structure or a tetraphenylmethane structure which were remained as the residue may be added diethyl ether followed by mixing, and then filtering the mixture using a glass filter, whereby they can be separated into the polymer residue, and a diethyl ether solution of the hypervalent iodine reactant having an adamantane structure or a tetraphenylmethane structure. The hypervalent iodine reactant having an adamantane structure or a tetraphenylmethane structure can be recovered by concentrating the diethyl ether solution. The process for recovering the hypervalent iodine reactant having an adamantane structure or a tetraphenylmethane structure is not limited to the foregoing examples, and each component can be separated by selecting an adequate solvent, making use of the difference in solubility of the polymer, the hypervalent iodine reactant having an adamantane structure or a tetraphenylmethane structure, the oxidizing agent not including a metal and the unreacted monomer, depending on the solvent type.

The electrically conductive resin composition of present invention is an electrically conductive resinous material containing the heterocycle-containing aromatic polymer (1) as a resin component. Only one type of the heterocycle-containing aromatic polymer (1) may be used alone, or two or more types may be used as a mixture. The electrically conductive resin composition of the present invention may further contain (i) a binder, (ii) an additive, (iii) a solvent, and the like to meet the object.

The binder (i) described above is not particularly limited, polyesters, poly(meth)acrylates, polyurethanes, polyvinyl acetates, polyvinylidene chloride, polyamides, polyimides, and copolymers constituted with at least two monomers selected from the group consisting of styrene, vinylidene chloride, vinyl chloride and alkyl (meth)acrylate, and the like are exemplified. The electrically conductive resin composition of the present invention contains the binder in an amount of preferably 1 to 5,000 parts by weight, and more preferably 10 to 3,000 parts by weight relative to 100 parts by weight of the heterocycle-containing aromatic polymer (1).

As the aforementioned additive (ii), a silane coupling agent for improving adhesiveness with the substrate or for improving durability of the coated film, as well as a leveling agent or a surfactant for improving coating properties are exemplified. Examples of the silane coupling agent include 3-glycidoxypropyltrimethoxysilane, 3-glycidoxypropyltriethoxysilane, 2-(3,4-epoxycyclohexyl)methyltrimethoxysilane, 2-(3,4-epoxycyclohexyl)methyltriethoxysilane, 2-(3,4-epoxycyclohexyl)ethyltrimethoxysilane, 2-(3,4-epoxycyclohexyl)ethyltriethoxysilane, (meth) acryloxytrialkoxysilane such as 3-methacryloxypropyltriethoxysilane, 3-methacryloxypropyltrimethoxysilane, 3-acryloxypropyltrimethoxysilane and 3-acryloxypropyltriethoxysilane; vinyltrimethoxysilane, vinyltriethoxysilane, p-styryltrimethoxysilane, and the like. These may be used alone, or a plurality of these may be used. When the silane coupling agent is compounded in the electrically conductive resin composition of the present invention, the silane coupling agent is contained in an amount of preferably 0.1 to 1,000 parts by weight, and more preferably 1 to 500 parts by weight relative to 100 parts by weight of the heterocycle-containing aromatic polymer (1). Examples of the surfactant include nonionic surfactants (e.g., polyoxyethylene alkylphenyl ethers, polyoxyethylene alkyl ethers, sorbitan fatty acid esters, fatty acid alkylolamides, and the like), fluorochemical surfactants (e.g., fluoroalkylcarboxylic acids, perfluoroalkylbenzenesulfonic acids, perfluoroalkyl quaternary ammoniums, perfluoroalkylpolyoxyethylene ethanols, and the like). The electrically conductive resin composition of the present invention contains preferably 0.01 to 30 parts by weight, and more preferably 0.05 to 10 parts by weight of the surfactant relative to 100 parts by weight of the electrically conductive resin composition.

Although the aforementioned solvent (iii) is not particularly limited, water, organic solvents (alcohols such as methanol, ethanol, 2-propanol, 1-propanol and n-butanol; ethylene glycols such as ethylene glycol, diethylene glycol, triethylene glycol and tetraethylene glycol; glycol ethers such as ethylene glycol monomethyl ether, diethylene glycol monomethyl ether, ethylene glycol dimethyl ether, ethylene glycol diethyl ether and diethylene glycol dimethyl ether; glycol ether acetates such as ethylene glycol monoethyl ether acetate, diethylene glycol monoethyl ether acetate and diethylene glycol monobutyl ether acetate; propylene glycols such as propylene glycol, dipropylene glycol and tripropylene glycol; propylene glycol ethers such as propylene glycol monomethyl ether, propylene glycol monoethyl ether, dipropylene glycol monomethyl ether, dipropylene glycol monoethyl ether, propylene glycol dimethyl ether, dipropylene glycol dimethyl ether, propylene glycol diethyl ether and dipropylene glycol diethyl ether; propylene glycol ether acetates such as propylene glycol monomethyl ether acetate, propylene glycol monoethyl ether acetate, dipropylene glycol monomethyl ether acetate and dipropylene glycol monoethyl ether acetate; N-methylformamide, N,N-dimethyl formamide, N-methylpyrrolidone, dimethylacetamide, dimethyl sulfoxide, acetone, acetonitrile, and toluene, xylene (o-, m-, or p-xylene), benzene, ethyl acetate, butyl acetate, methyl ethyl ketone, methyl isobutyl ketone, diethyl ether, diisopropyl ether, methyl-t-butyl ether, hexane, heptane, chloromethane (methyl chloride), dichloromethane (methylene chloride), trichloromethane (chloroform) and tetrachloromethane (carbon tetrachloride), etc.), as well as mixed solvents of water and any of these organic solvents (aqueous organic solvents), and mixed solvents of two or more kinds of the organic solvents are exemplified. The electrically conductive resin composition of the present invention contains preferably 100 to 5,000 parts by weight, and more preferably 500 to 3,000 parts by weight of the solvent relative to 100 parts by weight of the heterocycle-containing aromatic polymer (1). These may be used alone, or two or more thereof may be included in combination.

The electrically conductive resin composition of the present invention may be applied on, for example, a plastic substrate such as a polyester, acryl or polyurethane, or a glass substrate by any of a variety of coating processes to give a thickness of about 0.1 to 30 μm, followed by drying if necessary to permit curing, whereby an electrically conductive thin membrane or film can be obtained, and imparting antistatic property and functions as an electrode, electromagnetic shield, etc., to the substrate is enabled. Furthermore, in order to produce a solid electrolytic capacitor, an electrically-conductive layer required for solid electrolytic capacitors can be also formed by applying on the surface of a metal such as oxidized film-having aluminum, or sintered tantalum.

### EXAMPLES

Hereinafter, the present invention is specifically explained by way of Examples and Comparative Examples, but the present invention is not limited to these Examples.

### Example 1

To a 100 mL three-necked flask were charged dichloromethane (10 ml), 3,4-ethylenedioxythiophene (EDOT) (7) (4 mmol, 0.43 ml), bromotrimethylsilane (4 mmol, 0.53 ml), iodobenzene diacetate (PIDA) (6 mmol, 1.93 g), N-phenylpyrrole (8) (4 mmol, 572.7 mg), 1,1,1,3,3,3-hexafluoro-2-propanol (1 ml), and trifluoroacetic acid (4 mmol, 0.3 ml) under a nitrogen atmosphere at room temperature, and the mixture was stirred for 4 hrs. Four hours later, thereto was added saturated aqueous sodium bicarbonate (about 40 ml), and the mixture was subjected to liquid separating extraction using methylene chloride. After the organic layer was dried over Na SO₄ and filtered, the filtrate was concentrated under reduced pressure. Thus obtained crude product was purified using column chromatography to obtain 788.4 mg of an N-phenylpyrrole-EDOT coupling product (9).

¹H NMR (CDCl₃): 3.81-3.84 (2H, m), 3.99-4.01 (2H, m), 6.16 (1H, s), 6.33 (1H, dd, J=3.2, 2.7 Hz), 6.50 (1H, dd, J=3.2, 1.8 Hz), 6.90 (1H, dd, J=2.7, 1.8 Hz). 7.20-7.36 (5H, m)

¹³C NMR (CDCl₃): 64.20, 64.24, 98.21, 108.72, 109.21, 111.67, 123.47, 124.19, 125.66, 126.79, 128.56, 137.83, 140.33, 141.08. HRFABMS Calcd for C16H13NO2S (M); 283.0667. Found; 283.0675. EI-MS; 283.00 (100%), 186.00 (76.0%), 154.00 (35.0%), 77.00 (26.1%)

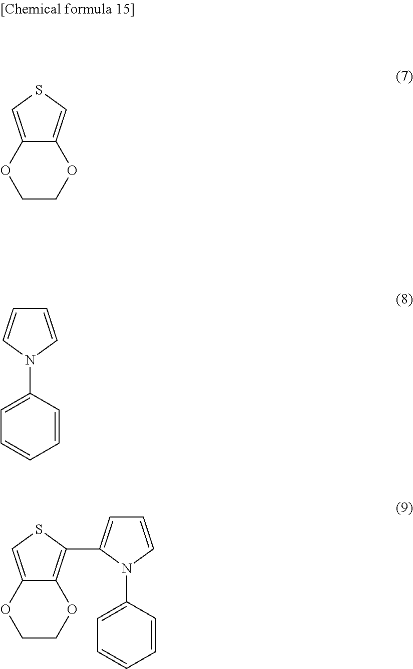

### Example 2

In a similar manner to Example 1 except that pyrrole (10) (4 mmol, 268.4 mg) was used in place of N-phenylpyrrole (8), and trifluoroacetic acid was not added in Example 1, 179.9 mg of a pyrrole-EDOT coupling product (11) was obtained.

¹H-NMR (CDCl₃): 4.21-4.25 (2H, m), 4.29-4.32 (2H, m), 6.11 (1H, s), 6.20-6.23 (1H, m), 6.31 (1H, s), 6.79 (1H, s), 9.10 (1H, bs). HR FABMS Calcd for C10H9NO2S (M); 207.0354. Found; 207.0357. EI-MS; 207.00 (99.5%), 151.00 (12.8%), 110.00 (100%)

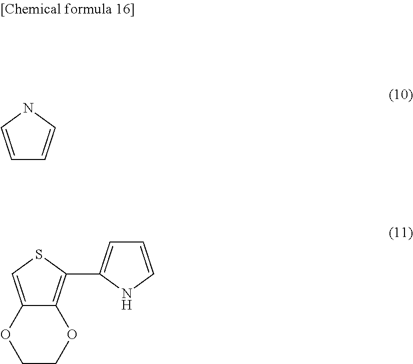

### Example 3

In a similar manner to Example 1 except that N-(4-fluorophenyl)pyrrole (12) (4 mmol, 644.7 mg) was used in place of N-phenylpyrrole (8) in Example 1, 940.0 mg of an N-(4-fluorophenyl)pyrrole-EDOT coupling product (13) was obtained.

¹H NMR (CDCl₃): 3.89-3.92 (2H, m), 4.04-4.06 (2H, m), 6.33 (1H, dd, J=3.5, 2.7 Hz), 6.50 (1H, dd, J=3.5 1.8 Hz), 6.86 (1H, dd, J=2.7, 1.8 Hz), 6.99-7.07 (2H, m), 7.18-7.24 (2H, m)

¹³C NMR (CDCl₃): 64.31, 64.33, 98.41, 108.49, 109.35, 111.67, 115.28, 115.57, 123.58, 124.49, 127.45, 127.57, 136.44, 137.89, 141.15, 159.81, 163.07

HRFABMS Calcd for C16H12FNO2S (M); 301.0573. Found; 301.0573. EI-MS; 301.00 (100%), 204.00 (88.1%), 172.00 (39.5%)

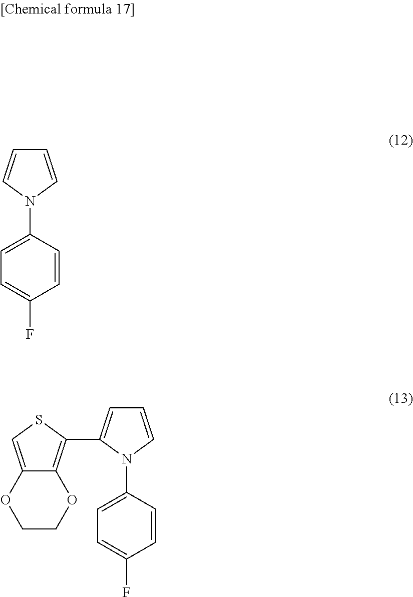

### Example 4

In a similar manner to Example 1 except that N-(4-methoxyphenyl)pyrrole (14) (4 mmol, 692.8 mg) was used in place of N-phenylpyrrole (8) in Example 1, 639.3 mg of an N-(4-methoxyphenyl)pyrrole-EDOT coupling product (15) was obtained.

¹H-NMR (CDCl₃): 3.82 (3H, s), 3.96-4.01 (2H, m), 4.07-4.10 (2H, m). 6.31 (1H, dd, J=3.6, 2.8 Hz), 6.53 (1H, dd, J=3.6, 1.8 Hz), 6.83 (1H, dd, J=2.8, 1.8 Hz), 6.86-6.90 (2H, m), 7.16-7.20 (2H, m).

HRFABMS Calcd for C17H15NO3S (M); 313.0773. Found; 313.0782.

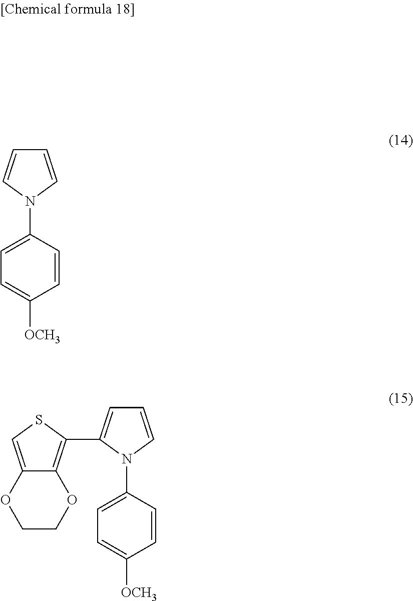

### Example 5

In a similar manner to Example 1 except that N-(4-trifluoromethylphenyl)pyrrole (16) (4 mmol, 844.7 mg) was used in place of N-phenylpyrrole (8) in Example 1, 744.8 mg of an N-(4-trifluoromethylphenyl)pyrrole-EDOT coupling product (17) was obtained.

¹H-NMR (CDCl₃): 3.77-3.79 (2H, m), 4.00-4.03 (2H, m), 6.38 (1H, dd, J=3.3, 2.7 Hz)., 6.49 (1H, dd, J=3.3, 1.8 Hz), 6.95 (1H, dd, J=2.7, 1.8 Hz), 7.34 (2H, d, J=8.2 Hz), 7.61 (2H, d, J=8.6 Hz). HRFABMS Calcd for C17H12F3NO2S (M); 351.0541. Found; 351.0536. EI-MS; 351.00 (77.4%), 254.00 (100%), 222.00 (41.1%)

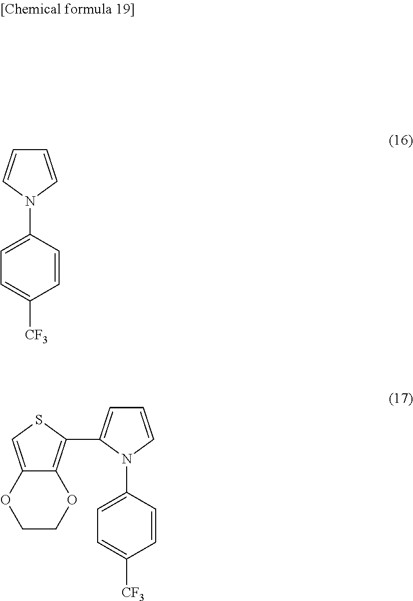

### Example 6

In a similar manner to Example 1 except that N-(4-carbomethoxyphenyl)pyrrole (18) (4 mmol, 804.9 mg) was used in place of N-phenylpyrrole (8) in Example 1, 764.7 mg of an N-(4-carbomethoxyphenyl)pyrrole-EDOT coupling product (19) was obtained.

¹H NMR (CDCl₃): 3.07 (2H, s), 3.84 (3H, s), 3.93 (2H, s), 6.16 (1H, s), 6.29 (1H, s), 6.89 (1H, s), 7.20 (2H, d, J=7.2 Hz), 7.94 (2H, d, J=7.2 Hz)

¹³C NMR (CDCl₃): 52.17, 64.20, 64.26, 98.80, 108.19, 110.11, 112.90, 123.23, 123.94, 124.58, 127.91, 130.17, 137.98, 141.18, 144.36, 166.47.

HRFABMS Calcd for C18H15NO4S (M); 341.0722. Found; 341.0739.

EI-MS; 341.00 (100%), 244.00 (41.8%), 212.00 (22.0%), 153.00 (14.7%)

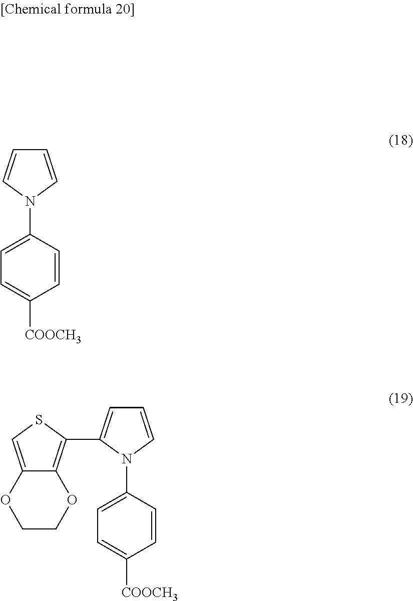

### Example 7

In a similar manner to Example 1 except that N-1-naphthylpyrrole (20) (4 mmol, 773.0 mg) was used in place of N-phenylpyrrole (8) in Example 1, 613.5 mg of an N-1-naphthylpyrrole-EDOT coupling product (21) was obtained.

¹H-NMR (CDCl₃): 3.89-3.95 (2H, m), 4.00-4.04 (2H, m), 5.90 (1H, s), 6.45 (1H, dd, J=3.6, 2.7 Hz), 6.76 (1H, dd, J=3.6, 1.8 Hz), 6.88 (1H, dd, J=2.7, 1.8 Hz), 7.36-7.51 (5H, m), 7.87-7.94 (2H, m). HRFABMS Calcd for C20H15NO2S (M); 333.0823. Found; 333.0823.

EI-MS; 333.00 (100%), 236.00 (55.4%), 204.00 (45.8%)

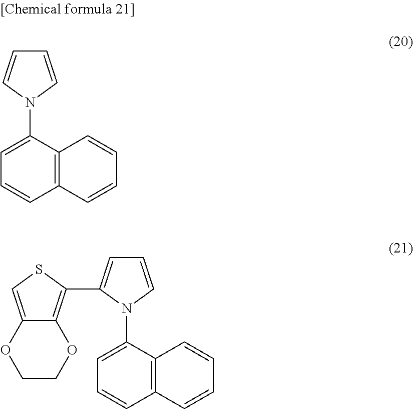

### Examples 8 to 14

Next, according to the compounding ratio shown in Table 1, the coupling products obtained in Examples 1 to 7 were each dissolved in diethylene glycol ethylmethyl ether (EDM), and thereafter p-toluenesulfonic acid iron (III) was added thereto followed by stirring for 1 min to obtain polymerizable compositions.

Subsequently, thus prepared polymerizable composition was applied on a glass plate (JIS R3202) using a No. 4 wire bar (amount of application: to give a thickness of about 9 μm in a wet state). Next, the composition was dried by air blowing at 60° C. for 10 min. After thus resulting thin membrane was immersed in pure water, it was dried at 100° C. for 1 min to obtain a thin membrane.

Next, surface resistivity of the obtained thin membrane was measured using Hiresta-UP (MCP-HT450) (manufactured by Mitsubishi Chemical Corporation) according to JIS K6911. In addition the film thickness of the obtained thin membrane was measured using a Stylus Surface Profilemeter Dektak 6M (manufactured by ULVAC, Inc.), and the electric conductivity was derived from the surface resistivity and the film thickness.

The total light transmittance of the obtained thin membrane was measured using a haze computer HGM-2B manufactured by Suga Test Instruments Co., Ltd., according to JIS K7150.

Furthermore, the solubility in solvents of the thin membrane obtained in the foregoing was examined. A sample in an amount of 1 mg taken by scraping the thin membrane using a razor from the glass plate described above was weighed into a 1 ml screw vial. Thereto was added 100 mg of a solvent (NMP(N-methylpyrrolidone), MEK (methyl ethyl ketone), or toluene), and shaken for 30 min to mix well. Several drops of thus obtained solution were placed on a 40 mm×50 mm cover glass, and further thereon was placed a cover glass of 40 mm×50 mm from above, whereby the solution was sandwiched. The appearance was observed by visual inspection, and the solubility in solvents was determined as in the following.

A: Soluble: the solvent being colored, having transparency, and no solid matter (insoluble matter) observed.
B: Partially soluble: solid matter (insoluble matter) observed, but the solvent being colored.
C: Insoluble: the solvent not being colored, and solid matter (insoluble matter) observed.

Moreover, as a marker of the weather resistance of the thin membrane obtained as described above, ultraviolet ray resistance was examined. More specifically, after the thin membrane was exposed to ultraviolet rays for 10 min, the surface resistivity of the thin membrane was determined.

In addition, heat resistance of the thin membrane obtained as described above was examined. More specifically, after the thin membrane was heated at 200° C. for 1 hour, the surface resistivity of the thin membrane was measured.

The results regarding the surface resistivity, the total light transmittance, the solubility in solvents, the ultraviolet ray resistance, and the heat resistance are shown in Table 2.

### Comparative Example 1

Next, 1.0 g of pyrrole (10) was dissolved in 13 g of diethylene glycol ethylmethyl ether (EDM), and thereafter 2.0 g of p-toluenesulfonic acid iron (III) was added thereto followed by stirring for 1 min to obtain a polymerizable composition.

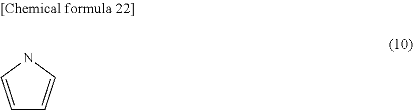

After a thin membrane was produced in a similar manner to that described above using the resulting polymerizable composition, tests on the surface resistivity, the total light transmittance, the solubility in solvents, the ultraviolet ray resistance, and the heat resistance were performed. Their results are shown in Table 2.

### Comparative Example 2

Next, 1.7 g of N-phenylpyrrole (8) was dissolved in 13 g of diethylene glycol ethylmethyl ether (EDM), and thereafter 2.0 g of p-toluenesulfonic acid iron (III) was added thereto followed by stirring for 1 min to obtain a polymerizable composition.

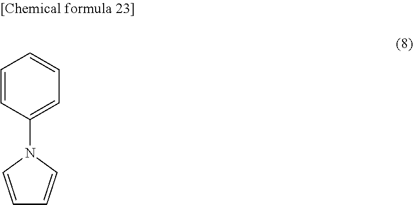

After a thin membrane was produced in a similar manner to that described above using the resulting polymerizable composition, tests on the surface resistivity, the total light transmittance, the solubility in solvents, the ultraviolet ray resistance, and the heat resistance were performed. Their results are shown in Table 2.

TABLE 1

| | Ex. 8 | Ex. 9 | Ex. 10 | Ex. 11 | Ex. 12 | Ex. 13 | Ex. 14 |
|---|---|---|---|---|---|---|---|
| Coupling product | Ex. 1 | Ex. 2 | Ex. 3 | Ex. 4 | Ex. 5 | Ex. 6 | Ex. 7 |
| Coupling product | 41.7 | 42.2 | 33.6 | 45.7 | 39.9 | 38.9 | 43.3 |
| (mg) | | | | | | | |
| EDM (mg) | 1,730 | 1,730 | 1,730 | 1,710 | 1,720 | 1,730 | 1,720 |
| p-toluenesulfonic | 200 | 200 | 200 | 200 | 200 | 200 | 200 |
| acid iron (III) (mg) | | | | | | | |

TABLE 2

| | Electric | Total light | | | | Ultraviolet ray | Heat |
|---|---|---|---|---|---|---|---|
| | conductivity | transmittance | Solubility in solvents | | | resistance | resistance |
| | [S/cm] | [%] | NMP | MEK | toluene | [S/cm] | [S/cm] |
| Ex. 8 | 1.25E−03 | 86.32 | A | B | B | 1.17E−04 | 1.20E−05 |
| Ex. 9 | 1.10E+01 | 89.72 | B | B | B | 8.93E+00 | 1.03E−02 |
| Ex. 10 | 1.18E−06 | 87.23 | A | A | B | 1.10E−06 | 3.15E−09 |
| Ex. 11 | 1.05E−04 | 84.94 | A | A | B | 3.62E−05 | 2.03E−06 |
| Ex. 12 | 2.52E−08 | 90.14 | A | A | B | 1.92E−09 | 3.99E−09 |
| Ex. 13 | 4.31E−07 | 89.76 | A | B | B | 1.06E−09 | 1.09E−09 |
| Ex. 14 | 1.17E−05 | 90.23 | A | B | A | 3.49E−06 | 4.65E−08 |
| Compar. | 3.43E−07 | 86.98 | C | C | C | 9.90E−11 | 9.80E−11 |
| Ex. 1 | | | | | | | |

| | |
|---|---|
| Compar. | Membrane formation failed |
| Ex. 2 | |

From Table 1, it is proven that the thin membranes obtained from the polymerizable compositions of Examples 8 to 14 had high electric conductivity and transparency comparative to or higher than the thin membrane obtained from the polymerizable composition of Comparative Example 1.

On the other hand, since the thin membrane of Comparative Example 1 did not exhibit favorable solubility in organic solvents such as NMP, MEK and toluene, it was difficult to evenly apply and to allow a film to be formed, suggesting inferior formation processability. To the contrary, it is proven that the thin membranes of Examples 8 to 14 exhibited better solubility in these organic solvents. Accordingly, it is revealed that due to capable of being uniformly dissolved in a broad range of organic solvents, they can be evenly coated as an electrically conductive coating material, and thus have favorable formation processability.

The thin membrane of Comparative Example 1 exhibited significantly elevated rate of increase in the surface resistivity after ultraviolet irradiation or after heating at 200° C., revealing that the electric conductivity is greatly deteriorated when used in severe environments. In contrast, it is proven that the thin membranes of Example 8 to 14 exhibited lower rate of increase in the surface resistivity, suggesting exhibiting stable electric conductivity without being affected by environment.

Accordingly, it is proven that the heterocycle-containing aromatic compound of the present invention provides an electrically conductive polymer having well balanced electric conductivity, heat resistance, weather resistance, transparency, and formation processability.

Moreover, in Comparative Example 2, the polymerization reaction is difficult to proceed, and the film formation failed. To the contrary, according to the coupling product of Example 1 having a similar N-phenylpyrrole skeleton, the polymerization easily proceeded, thereby capable of providing an electrically conductive thin membrane; therefore, even with a monomer having a low reactivity, conversion into the heterocycle-containing aromatic compound of the present invention enables introduction into a polymer skeleton, suggesting possibility of permitting broader design discretion of electrically conductive polymers.

### Example 15

Into a 200 mL eggplant flask were charged 25 g of ion exchanged water, 2.6 g of a 12.8% by mass aqueous polystyrene sulfonic acid solution, and 40 mg (0.0932 mmol) of PIFA dissolved in 1,1,1,3,3,3-hexafluoro-2-propanol (3 ml). Subsequently, 275 mg (0.932 mmol) of the EDOT-N-phenylpyrrole coupling product (9) was added thereto after dissolving in 3 ml of acetonitrile, and further 1.8 g of a 10.9% by mass aqueous peroxodisulfuric acid solution was added to the mixture. Thereafter, the mixture was stirred at room temperature (about 25° C.) for 24 hrs, and disappearance of the EDOT-N-phenylpyrrole coupling product (9) was confirmed by HPLC to obtain 35 g of a water dispersion of polystyrene sulfonic acid-doped poly(EDOT-N-phenylpyrrole) (solid content: 1.7%).

With respect to this water dispersion of poly(EDOT-N-phenylpyrrole), since polystyrene sulfonic acid was used as a dopant, measurement of the molecular weight of the poly(EDOT-N-phenylpyrrole) itself by GPC determination was difficult. However, it is clear that the poly(EDOT-N-phenylpyrrole) was generated on the ground that the EDOT-N-phenylpyrrole coupling product (9) completely disappeared in the polymerization reaction, and that test results of the electrically conductive resin composition of the present invention shown below suggested the electric conductivity exhibited by the water dispersion.

### Example 16

Into a 2,000 mL three-necked flask were charged 3.10 g (10.9 mmol) of the EDOT-N-phenylpyrrole coupling product (9), 410 g of ion exchanged water, 253 g of a 12.8% by mass aqueous polystyrene sulfonic acid solution, and 16.5 g (0.41 mmol) of a 1% aqueous sulfuric acid iron (III) solution. Subsequently, 11.8 g (5.7 mmol) of a 10.9% by mass aqueous peroxodisulfuric acid solution was added to the mixture. Thereafter, the mixture was stirred at room temperature (about 25° C.) for 24 hrs, and disappearance of the EDOT-N-phenylpyrrole coupling product (9) was confirmed by HPLC to obtain 650 g of a water dispersion of polystyrene sulfonic acid-doped poly(EDOT-N-phenylpyrrole) (solid content: 1.1%).

With respect to this water dispersion of poly(EDOT-N-phenylpyrrole), since polystyrene sulfonic acid was used as a dopant, measurement of the molecular weight of the poly(EDOT-N-phenylpyrrole) itself by GPC determination was difficult. However, it is clear that the poly(EDOT-N-phenylpyrrole) was generated on the ground that the EDOT-N-phenylpyrrole coupling product (9) completely disappeared in the polymerization reaction, and that test results of the electrically conductive resin composition of the present invention shown below suggested the electric conductivity exhibited by the water dispersion.

### Example 17

Into a 30 mL eggplant flask were charged 100 mg (0.48 mmol) of the EDOT-pyrrole coupling product (11) and 10 mL of methylene chloride, and the mixture was stirred under a nitrogen atmosphere at room temperature (about 25° C.). Then, 213.4 mg of trimethylsilyl trifluoromethanesulfonate was added thereto, and thereafter, 15.4 mg of phenyliodin diacetate (PIDA), 138 mg (0.72 mmol) of metachloroperbenzoic acid (60%), and 27.3 μl (0.72 mmol) of acetic acid were added. Thereafter, the mixture was further stirred for 12 hrs. After confirming disappearance of the EDOT-pyrrole coupling product (11) by HPLC, the reaction solution was concentrated under reduced pressure, and 20 mL of methanol was added to the residue. Subsequently, black insoluble matter was filtered off with a glass filter, and washed with 30 ml of methanol. Additional washing with 30 ml of hexane gave 85.4 mg of poly(EDOT-pyrrole).

Determination of the molecular weight of the resulting poly(EDOT-pyrrole) revealed the weight average molecular weight (Mw) of 7,137, the number average molecular weight (Mn) of 7,072, and the ratio Mw/Mn of the weight average molecular weight to the number average molecular weight being 1.009.

### Example 18

Into a 300 mL three-necked flask were charged 0.6 g (2.9 mmol) of the EDOT-pyrrole coupling product (11), 140 g of ion exchanged water, 65 g of a 12.8% by mass aqueous polystyrene sulfonic acid solution, and 5.0 g (0.13 mmol) of a 1% aqueous sulfuric acid iron (III) solution. Subsequently, 7.3 g (3.3 mmol) of a 10.9% by mass aqueous sodium peroxodisulfate solution was added to the mixture. Thereafter, the mixture was stirred at room temperature (about 25° C.) for 24 hrs, and disappearance of the EDOT-pyrrole coupling product (11) was confirmed by HPLC to obtain 200 g of a water dispersion of polystyrene sulfonic acid-doped poly(EDOT-pyrrole) (solid content: 0.9%).

With respect to this water dispersion of poly(EDOT-pyrrole), since polystyrene sulfonic acid was used as a dopant, measurement of the molecular weight of the poly(EDOT-pyrrole) itself by GPC determination was difficult. However, it is clear that the poly(EDOT-pyrrole) was generated on the ground that the EDOT-pyrrole coupling product (11) completely disappeared in the polymerization reaction, and that test results of the electrically conductive resin composition of the present invention shown below suggested the electric conductivity exhibited by the water dispersion.

### Examples 19 to 22

First, the solubility in solvents of the polymer obtained in the aforementioned Examples 15 to 18 was examined. The polymers obtained in Examples 15, 16 and 18 in the form of a water dispersion were applied on a glass substrate using a No. 4 wire bar, and dried by air blowing at 100° C. for 2 min. Samples of the polymer were prepared by taking the thin membrane of the obtained polymer by scraping with a razor. Since the polymer of Example 17 was solid, it was directly subjected to the evaluation. These samples of the polymers of Examples 15 to 18 in an amount of 1 mg were weighed into a 1 ml screw vial, and thereto was added 100 mg of a solvent (NMP(N-methylpyrrolidone), MEK (methyl ethyl ketone), or toluene). The samples were mixed well by shaking for 30 min. Several drops of thus obtained solution were placed on a 40 mm×50 mm cover glass, and further thereon was placed a cover glass of 40 mm×50 mm from above, whereby the solution was sandwiched. The appearance was observed by visual inspection, and the solubility in solvents was determined as in the following.

A: Soluble: the solvent being colored, having transparency, and no solid matter (insoluble matter) observed.
B: Partially soluble: solid matter (insoluble matter) observed, but the solvent being colored.
C: Insoluble: the solvent not being colored, and solid matter (insoluble matter) observed.

Next, using the polymers obtained in Examples 15 to 18, each source material was mixed at the compounding ratio shown in Table 3. After completely admixed, each compounded matter was filtered through a 0.5 μm membrane filter to remove insoluble matter. Accordingly, the electrically conductive resin compositions of the present invention were prepared, and thin membranes were formed according to the following procedure. Thus prepared electrically conductive resin composition was applied on a glass plate (JIS R3202) using a No. 4 wire bar (amount of application: to give a thickness of 9 μm in a wet state). Next, the composition was dried by air blowing at 100° C. for 2 min to give a thin membrane. The thickness of the resulting thin membranes was as shown in Table 4.

Next, surface resistivity of the obtained thin membrane was measured using Hiresta-UP (MCP-HT450) (manufactured by Mitsubishi Chemical Corporation) according to JIS K6911.

The total light transmittance of the obtained thin membrane was measured using a haze computer HGM-2B manufactured by Suga Test Instruments Co., Ltd., according to JIS K7150.

Further, weather resistance of the thin membrane obtained as described above was examined. More specifically, after the thin membrane was exposed to ultraviolet rays for 10 min, the surface resistivity of the thin membrane was determined.

In addition, heat resistance of the thin membrane obtained as described above was examined. More specifically, after the thin membrane was heated at 200° C. for 1 hour, the surface resistivity of the thin membrane was measured.

The results regarding the film thickness, the surface resistivity, the total light transmittance, the solubility in solvents, the weather resistance, and the heat resistance of the obtained thin membrane are shown in Table 4.

### Comparative Example 3

Into a 50 mL eggplant flask were charged 67 mg (1 mmol) of pyrrole (10) and 10 mL of acetonitrile. Then, 644 mg (2 mmol) of PIDA and 392 mg (2 mmol) of paratoluenesulfonic acid were added thereto, and the mixture was stirred at room temperature for 20 hrs. After disappearance of the pyrrole (10) was confirmed by HPLC, insoluble matter of this reaction solution was filtered off with a glass filter. Then, insoluble matter was washed with methanol, and further washed with n-hexane to obtain 110 mg of fine powdery polypyrrole. Since thus obtained pyrrole was insoluble in various types of solvents, it was impossible to determine the molecular weight by GPC.

### Comparative Example 4

Polypyrrole obtained in Comparative Example 3 in an amount of 34 mg was dispersed in 2,700 mg of chloroform, and thereto were added 150 mg of a polyester binder and 32 mg of a surfactant. Further, 115 mg of N-methylpyrrolidone was added thereto as a solvent to prepare an electrically conductive resin composition. With respect to this electrically conductive resin composition, various types of tests were performed in similar procedures to Examples 19 to 22. The results are shown in Table 4.

TABLE 3

| | Ex. 19 | Ex. 20 | Ex. 21 | Ex. 22 | Compar. Ex. 4 |
|---|---|---|---|---|---|
| Electrically conductive | Ex. 15 | Ex. 16 | Ex. 17 | Ex. 18 | Compar. Ex. 3 |
| polymer | | | | | |
| Electrically conductive | 2,700 | 2,700 | 27 | 2,700 | 34 |
| polymer (mg) | | | | | |
| Solvent 1 | 80% ethanol | 80% ethanol | NMP | NMP | chloroform |
| Solvent 1 (mg) | 3,000 | 3,000 | 3,000 | 119 | 2,700 |
| Solvent 2 | NMP | NMP | — | — | NMP |
| Solvent 2 (mg) | 118 | 121 | — | — | 115 |
| Polyester binder (mg) | 148 | 146 | — | 151 | 150 |
| Silane coupling agent | — | — | 41 | — | — |
| (mg) | | | | | |
| Surfactant (mg) | 29 | 33 | 30 | 29 | 32 |

With respect to the electrically conductive polymers of Examples 19, 20 and 22, the amount charged in the form of the water dispersion liquid is shown. In regard to the solvent, 80% ethanol means aqueous ethanol containing 20% water, and NMP indicates N-methylpyrrolidone. Gabusen® ES-901A manufactured by Nagase ChemteX Corporation was used as the polyester binder; a fluorochemical surfactant Plas coat RY-2 manufactured by Goo Chemical Company, Ltd. was used as the surfactant; and Silquest A-187 manufactured by Momentive Performance Materials Japan Inc. was used as the silane coupling agent.

TABLE 4

| | Film | Surface | Total light | | | | Ultraviolet ray | Heat |
|---|---|---|---|---|---|---|---|---|
| | thickness | resistivity | transmittance | Solubility in solvents | | | resistance | resistance |
| | [μm] | [Ω/□] | [%] | NMP | MEK | toluene | [Ω/□] | [Ω/□] |
| Ex. 19 | 0.17 | 1.17E+10 | 86.35 | B | B | B | 5.07E+11 | 2.45E+10 |
| Ex. 20 | 0.15 | 5.16E+08 | 87.22 | B | B | B | 3.29E+09 | 1.76E+09 |
| Ex. 21 | 0.17 | 2.87E+10 | 91.54 | A | B | B | 3.01E+11 | 1.19E+12 |
| Ex. 22 | 0.27 | 2.56E+08 | 85.07 | B | B | B | 3.89E+09 | 2.18E+10 |
| Compar. | 0.31 | 8.79E+12 | 81.90 | C | C | C | 1.00E+14 | 1.01E+14 |
| Ex. 4 | | | | | | | | |

From Table 4, it is proven that the thin membranes obtained from the electric conductive compositions of Examples 19 to 22 compounded using the electrically conductive polymer obtained in Examples 15 to 18 had higher electric conductivity and transparency than the thin membrane obtained from the electric conductive composition of Comparative Example 4.

In contrast to the polymer of Comparative Example 4 not exhibiting favorable solubility in organic solvents such as NMP, MEK and toluene, the polymers of Examples 19 to 22 were revealed to be soluble in any of these organic solvents. It is proven that due to being soluble in a broad range of organic solvents, they can be evenly coated as an electrically conductive coating material, and thus are superior in formation processability.

The thin membrane of Comparative Example 4 exhibited significantly elevated rate of increase in the surface resistivity after ultraviolet irradiation or after heating at 200° C., indicating that the electric conductivity is lost when used in severe environments. In contrast, it is proven that the thin membranes of Example 19 to 22 maintained the electric conductivity even after ultraviolet irradiation or after heating at 200° C., suggesting having stable electric conductivity without being affected by environment.

### INDUSTRIAL APPLICABILITY

According to the present invention, a novel heterocycle-containing aromatic compound, a simple method for production of the same, a polymerizable composition containing the compound, a novel heterocycle-containing electrically conductive polymer, a simple method for production of the same, and an electrically conductive resin composition containing the polymer are provided. The heterocycle-containing aromatic compound or the composition of the present invention is polymerized by heat or a radioactive ray, and can provide an electrically conductive polymer or cured product. This polymer or cured product can maintain the transparency, electric conductivity, heat resistance, and the like at high levels.

Conventional electrically conductive polymers produced using a single heterocyclic aromatic compound as a monomer generally have poor solubility in organic solvents, and low transparency; therefore, industrially applicable field of them have been limited. According to the present invention, a variety of types of polymers can be synthesized that are superior in stability, solubility in solvents, transparency, electric conductivity and the like, by appropriately selecting structures of two different aromatic compounds. In addition, the method for production of the same is superior as an industrial production method since it is more convenient as compared with electrolytic polymerization methods, and large-scale production is enabled.

Therefore, according to the present invention, electrically conductive polymers are provided which can be used for electrically conductive materials of various types of electronic parts (electrically conductive films, solid electrolytic capacitors, transparent electrodes used in liquid crystal panels, touch panels, etc., and the like) and solar cells, as well as antistatic agents, and the like.

1. A heterocycle-containing aromatic compound represented by the following general formula (1):
A-B (1)
(wherein, A represents a substituted or unsubstituted thiophene ring group, or a substituted or unsubstituted pyrrole ring group; B represents a substituted or unsubstituted hydrocarbon aromatic ring group, a substituted or unsubstituted thiophene ring group, or a substituted or unsubstituted pyrrole ring group; and the ring represented by A and the ring represented by B are directly linked; however, A and B represent structures that are different from each other).

2. The compound according to claim 1 represented by the following general formula (2) and constituted by binding between the thiophene ring group and the pyrrole ring group: 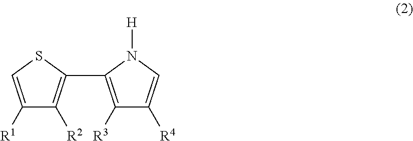
(wherein, R¹ and R² each independently represent a hydrogen atom or an organic group and at least one represents an organic group, and R³ and R⁴ each independently represent a hydrogen atom or an organic group; alternatively, R¹ and R² both represent a hydrogen atom, and R³ and R⁴ each independently represent an organic group; when both R¹ and R² represent an organic group, these may bind with each other to form a ring structure; whereas when both R³ and R⁴ represent an organic group, these may bind with each other to form a ring structure.)

3. The compound according to claim 2, wherein in the formula (2), R¹ and R² each independently represent an organic group, which bind with each other to form a ring structure, and/or R³ and R⁴ each independently represent an organic group, which bind with each other to form a ring structure.

4. The compound according to claim 2, wherein in the formula (2), R¹ and R² each independently represent an organic group, which bind with each other to form a ring structure.

5. The compound according to claim 2, wherein in the formula (2), R¹ and R² bind with each other to form an ethylenedioxy group.

6. The compound according to claim 2 represented by the following formula:

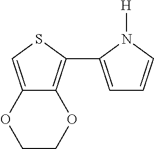

7. The compound according to claim 1 represented by the following general formula (3) and constituted by binding between the thiophene ring group and the N-substituted pyrrole ring group: 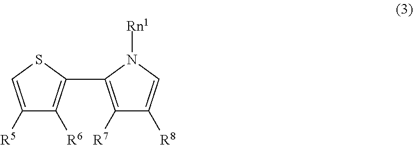
(wherein, R⁵ and R⁶ each independently represent a hydrogen atom or an organic group and at least one represents an organic group, and R⁷ and R⁸ each independently represent a hydrogen atom or an organic group; alternatively, R⁵ and R⁶ both represent a hydrogen atom, and R⁷ and R⁸ each independently represent an organic group; when both R⁵ and R⁶ represent an organic group, these may bind with each other to form a ring structure; whereas when both R⁷ and R⁸ represent an organic group, these may bind with each other to form a ring structure; and Rn¹ represents an organic group.)

8. The compound according to claim 7, wherein in the formula (3), R⁵ and R⁶ each independently represent an organic group, which bind with each other to form a ring structure, and/or R⁷ and R⁸ each independently represent an organic group, which bind with each other to form a ring structure.

9. The compound according to claim 7, wherein in the formula (3), R⁵ and R⁶ each independently represent an organic group, which bind with each other to form a ring structure.

10. The compound according to claim 7, wherein in the formula (3), R⁵ and R⁶ bind with each other to form an ethylenedioxy group.

11. The compound according to claim 7 represented by the following formula: 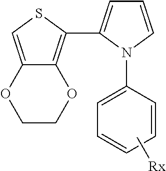
(wherein, Rx represents a hydrogen atom, an organic group or a halogen atom.)

12. The compound according to claim 7 represented by the following formula:

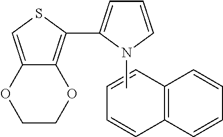

13. The compound according to claim 1 represented by the following general formula (4) and constituted by binding between the pyrrole ring group and the N-substituted pyrrole ring group: 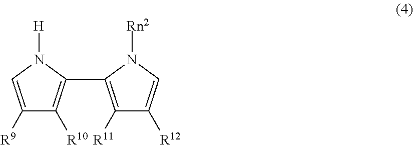
(wherein, R⁹ and R¹⁰ each independently represent a hydrogen atom or an organic group and at least one represents an organic group, and R¹¹ and R¹² each independently represent a hydrogen atom or an organic group; alternatively, R⁹ and R¹⁰ both represent a hydrogen atom, and R¹¹ and R¹² each independently represent an organic group; when both R⁹ and R¹⁰ represent an organic group, these may bind with each other to form a ring structure; whereas when both R¹¹ and R¹² represent an organic group, these may bind with each other to form a ring structure; and Rn² represents an organic group.)

14. The compound according to claim 13, wherein in the formula (4), R⁹ and R¹⁰ each independently represent an organic group, which bind with each other to form a ring structure, and/or R¹¹ and R¹² each independently represent an organic group, which bind with each other to form a ring structure.

15. The compound according to claim 1 represented by the following general formula (5) and constituted by binding between two different N-substituted pyrrole rings that are different from each other: 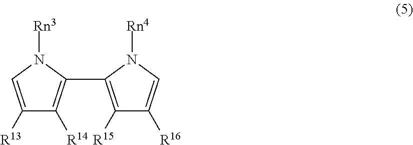
(wherein, R¹³ and R¹⁴ each independently represent a hydrogen atom or an organic group and at least one represents an organic group, and R¹⁵ and R¹⁶ each independently represent a hydrogen atom or an organic group; alternatively, R¹³ and R¹⁴ both represent a hydrogen atom, and R¹⁵ and R¹⁶ each independently represent an organic group; when both R¹³ and R¹⁴ represent an organic group, these may bind with each other to form a ring structure; whereas when both R¹⁵ and R¹⁶ represent an organic group, these may bind with each other to form a ring structure; Rn³ and Rn⁴ each independently represent an organic group; however, a combination of R¹³, R¹⁴ and Rn³, should not be identical to a combination of R¹⁵, R¹⁶ and Rn⁴.)

16. The compound according to claim 15, wherein in the formula (5), R¹³ and R¹⁴ each independently represent an organic group, which bind with each other to form a ring structure, and/or R¹⁵ and R¹⁶ each independently represent an organic group, which bind with each other to form a ring structure.

17. The compound according to claim 1 represented by the following general formula (6) and constituted by binding between a 3,4-ethylenedioxythiophene ring group and a benzene ring group. 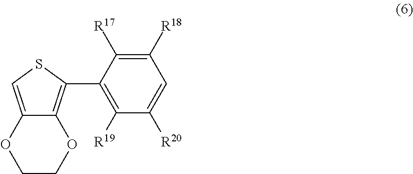
(wherein, R¹⁷ and R¹⁸ each independently represent a hydrogen atom or an organic group; and at least one represents an organic group; and R¹⁹ and R²⁰ each independently represent an organic group.)

18. A method for producing a heterocycle-containing aromatic compound represented by the following general formula (1):
A-B (1)
the method comprising allowing a compound represented by the formula of: A-H to be coupled with a compound represented by the formula of: B-H in the presence of a hypervalent iodine reactant,
(in each formula, A represents a substituted or unsubstituted thiophene ring group, or a substituted or unsubstituted pyrrole ring group; B represents a substituted or unsubstituted hydrocarbon aromatic ring group, a substituted or unsubstituted thiophene ring group, or a substituted or unsubstituted pyrrole ring group; the ring represented by A and the ring represented by B are directly linked; however, A and B represent structures that are different from each other.)

19. A polymerizable composition comprising the heterocycle-containing aromatic compound according to claim 1, and a dopant.

20. An electrically conductive polymer obtained by oxidative polymerization of the heterocycle-containing aromatic compound according to claim 1 as a monomer.

21. The electrically conductive polymer according to claim 20, wherein the oxidative polymerization is carried out by a chemical polymerization method using an oxidizing agent.

22. A method for producing an electrically conductive polymer comprising carrying out oxidative polymerization of the heterocycle-containing aromatic compound according to claim 1 as a monomer by a chemical polymerization method using an oxidizing agent.

23. The method according to claim 22, wherein the oxidizing agent is a hypervalent iodine reactant.

24. An electrically conductive resin composition comprising the electrically conductive polymer according to claim 20.
